SEQUENCE LISTING (11 sequences)

SEQ ID NO: 1 - protein, 396 aa, synthetic construct
  REGION 1..396: note = Pantoea agglomerans
MNKGENMNLEMWVQNISATFPWYAQLLRDKQADLSRLESLPLITEELLTQHYYHAENSFPGEHHSYLTSGTTSGKRKRIFYSDNDQRIYLQQRMDIIRDFCGEGHTRACADLGTGHAAATAGEIFQAMGCDVELIDFTRPIEQHIEVLNRFKPDIFFTMPMILDSLIATGKLDFQPKRIILLGDVASLNWQNKVADYFHIQPAQVLDLFGSIEIGSIAFYNHAQKRYQFDSYVRPEVVPVQSLYPGAKYGGDGGILLLTSFAREYFPAVRFVTNDLIEGFAQENVGGRTVYTYQRCLGRFAGEFKHGEKINLSDISDALANNLPYHKYDLADHEGGLVIRIAAKSIPTEVIEAIKHDLLARNPDIAQMISSGLVGDIRIQCVDAQEITGNVSKRRY

SEQ ID NO: 2 - protein, 530 aa, synthetic construct
  REGION 1..530: note = Streptomyces thioluteus
MAEFNEMKTTGDKLYHPELRTLVETARFHAERQPETPAILFEGGTTTYGQLHEESNRIAHAIRAAGAAPGTRVAYLGKESAHYYEILFGCAKSGTVLVPINWRLTAHEVSHILQDSGTELLFLEEEFAPILDRLPDGPPATVVRLGVPEGFAAWKAAHPATEPDVDAGPDTPFAQIYTSGTTGLPKGVVLAHRSCFAIRDALASEGLEWIDWRAGDVALVGIPGFHVGGLWWATQNFNAGVTVVVMRAFAAHAAVELIRDLGVTTACVVPAMLRLMLAEPGITPEHFTTLRKIVYGGSPISEALLEESLAMFGSEFAQIYGLTETGNTACCLPPSAHVPGSPRMKAAGHPYPGIRAKVIDAEGRELPTGSVGEVCLHTPARMVEYWGLPDKTAETLVDGWIHTGDAGYVDEDGYVFISDRIKDAVIVAGENVYPAEIENVLEGHPGVADAVVVGAPDERWGETVHAFVVPAPGHEPTPRDLHRFLVDRLASFKLPAAYEFIDEVPRNPSGKILRRELRERFWSGSDRKVN

SEQ ID NO: 3 - protein, 602 aa, Bacillus thuringiensis
MKHAKQIAEHATIQSFLNCYLRETGSGEWITEDKRIEDIFYHSFQRDTCSTYLCCRLSAQNITLYGEVIYKSPTDRHLFGEQFYYQMGDSNSVMKADYVTVITFLIKEMSINYGEGTNPAELMLRVIRSCQNIEEFTKERKEDTSALYGFHTSFIEAEQSLLFGHLTHPTPKSRQGILEWKSAMYSPELKGECQLHYFRAHKSIVNEKSLLLDSTTVILKEELRNDEMVSKEFISKYCNEDEYSLLPIHPLQAEWLLHQPYVQDWIEQGVLEYIGPTGKCYMATSSLRTLYHPDAKYMLKFSFPVKVTNSMRINKLKELESGLEGKAMLNTAIGEVLKKFPGFDFICDPAFITLNYGTQESGFEVIIRENPFYSEHADDATLIAGLVQDAIPGERTRLSNIIHRLADLESRSCEEVSLEWFRRYMNISLKPMVWMYLQYGVALEAHQQNSVVQLKDGYPVKYYFRDNQGFYFCNSMKEMLNNELAGIGERTGNLYDDYIVDERFRYYLIFNHMFGLINGFGTAGLIREEILLTELRTVLESFLPYNREPSTFLRELLEEDKLACKANLLTRFFDVDELSNPLEQAIYVQVQNPLVREVAVRS

SEQ ID NO: 4 - protein, 602 aa, synthetic construct; identical to SEQ ID NO: 3
  REGION 1..602: note = Bacillus thuringiensis serovar monterrey (BGSC 4AJ1)

SEQ ID NO: 5 - protein, 627 aa, Pseudomonas fluorescens
MTTLTSQALGQAADLVMQDLVDCLLAEHFFGIGPAPLVTIDDWQQAHPAAPTLTGVTARQRIWDWCYDPSEQRFISIALRQGITQQWEKVPGTPVLARQEEQWTVLSPEDFMKRAFGSMAERFKDNDKGLALFLDVLRTSVAQTALSLSHAVDERDLMAQAPATFFRIMEQWASLRDRPFHPLAKAKQGLDDEEYQRYQAEFARPVALNWVAVDHALLQCGEGVGDLAQCYPAQYLLPEPLQARLAGEMQARGLADSHIALPVHPWQWEHVLDKQLGDAFAKGDCQRLDFNEGDFFATSSLRSMTPCFDSAHYLKLPMAIHSLGASRYLPAVKMINGGLSEALLHEVLDKDATLRQSLHLCDEGKWWAFMPPQATLFDEAPRHLSAMVRGYPAALLADADTRLLPMAALGTPLPGSNRHFFDDWMQYRNLPAGAESVMALFRELAHSFFDINLRMFRLGMLGEVHGQNAVLVWRDGHAEGLLLRDHDSLRIFVPWLERNGMADPVYRIKKGHANTLYHERPEDLLFWLQTLGIQVNVRAIMDTLAQVYSIPVSGLWRVLREVLDQLIETIDFDDEARAMIRYQLFDAPDWPQKLLLTPMIERAGGPGSMPFGKGEVVNPFRRLSPEA

SEQ ID NO: 6 - protein, 595 aa, synthetic construct
  REGION 1..595: note = Streptomyces coelicolor A3(2)
MSLADAVAHLTPERWEEANRLLVRKALAEFTHERLLTPEREPDDGGGQTYVVRSDDGQTAYRFTATVRALDHWQVDAASVTRHRDGAELPLAALDFFIELKQTLGLSDEILPVYLEEISSTLSGTCYKLTKPQLSSAELARSGDFQAVETGMTEGHPCFVANNGRLGFGIHEYLSYAPETASPVRLVWLAAHRSRAAFTAGVGIEYESFVRDELGAATVDRFHGVLRGRGLDPADYLLIPVHPWQWWNKLTVTFAAEVARGHLVCLGEGDDEYLAQQSIRTFFNASHPGKHYVKTALSVLNMGFMRGLSAAYMEATPAINDWLARLIEGDPVLKETGLSIIRERAAVGYRHLEYEQATDRYSPYRKMLAALWRESPVPSIREGETLATMASLVHQDHEGASFAGALIERSGLTPTEWLRHYLRAYYVPLLHSFYAYDLVYMPHGENVILVLADGVVRRAVYKDIAEEIAVMDPDAVLPPEVSRIAVDVPDDKKLLSIFTDVFDCFFRFLAANLAEEGIVTEDAFWRTVAEVTREYQESVPELADKFERYDMFAPEFALSCLNRLQLRDNRQMVDLADPSGALQLVGTLKNPLAGR

SEQ ID NO: 7 - protein, 516 aa, synthetic construct
  REGION 1..516: note = Actinomycetales bacterium mxb001
MADGAMALLHDGLARWAATMPDEEAVAFGDHRLSWAQWHERIRRLTGALVARGIGAGDRVAVIDRNHLASLDMILAAGALGAITVLPNWRLKDAELTYVLGDCSPRLVVLGAAFVDREELIRMAAPSIEHVVHLDDDYESWLSSGTPTSPAADVRPDTPVLVIYTSGTTGAAKGVVFSHSALIANAEVSALTAHAEPGDRALTSMPLFHIGGVGAALAAVESGLPQTILADPTPDAIVQAIEQGCTRTFLVPAVIDRMLESGQRERQALASLSLLAYGGSPCPRPILEAALAAMPDTDIVQVYGMTELCGSVTQLSNTAHRDSDHRERLAAAGQLVDHVDVRIVDPTTLLDVSPGDTGELWFRTPKRMQGYLGQPEATAQVIVDGGWLRTGDIGRMDEEGFIFIVDRLKDIIITGGENVFSPEVESVVAEHPSVAEVAVIGMPDPTMGERVTAVVTAAQGKTVDPDDVITFTRTRLAGFKCPRTVVVVDELPRNASGKVLKHELRTSIAASNVGAS

SEQ ID NO: 8 - protein, 523 aa, synthetic construct
  REGION 1..523: note = Lysinibacillus sp.
MNAYLLLSIASSIAPEQEILTFFKRRLTYGQLSKRVGALASRLAADGVKRGDRVGIVSTNSPEVVESFFAAFQLGATIVPINYRAKPEELAYMLRDSGIKVLLIESRYVQSIEQFLTNPMCNIDKTIVIGDDAQGYELYETYIANNEPIIDFADIEEDDIAILLYTSGTTSLPKGVMLTYTQLTNYVMGHTEAATGLPNGASLTCVPNYHVAGATSICNCIYGGRRIVLIRQFEADTWLDTLEQEQVTHAFLVPTMLKRIIDHPRFGQTDFSSLQSLSYGAAPMPFPIIRQAIEMFPLSVDFANAFGMTETTSTVAVLGPDDHKLVGTPEEIARKIERLHSVGKPLPNVDIKILDDSQHELPPNEIGNVYIKTAKAMKGYWNRPDESQETLIDGWINTKDMGYLDEDGYLFLKGRNSDMIIRGGENIAPQEIENVLIAHPEISDAAVIGVPSVEWGEEILAVIIPSNALAPPAIADITAYCRERLASFKCPRIIEFATELPRTSSGKLLKRDLRDQYTTNLVE

SEQ ID NO: 9 - protein, 536 aa, synthetic construct
  REGION 1..536: note = Smithella sp. PtaU1.Bin162
MVANPYWSLEGIEEIEENNYKLRIHKNRPHTIDQAFRNTVKKYPMKEAIVAGNERWTYAKYDEKVDRVASALVEKFGIQPGDRVALLMNNHILWAVTFLAIARAGAISVPLNSRLQGKEILYQMNNSQTRLLIFDQEYFNRIQPFREEFRTIQTFVINGLCDPEGWVDYRELETYPSRNINVQRDETDGASILYTSGTTGLPKGALLTNRNILSIAVAVIDMLQFDKDDRMLIFIPLFHVTGLVGMLVAMFVTGGTLIILPGFKRHEIPQIIQEEKITATIGVPATFILIMESPNYQKYSRETMRCIVYGGAPSPPDMHQQLRDTLPKDCLLVEAYGLTESSSNVSIFPITKDRKVYNKYGSIGVVNPVADIRIVDEQGNDLPDGEIGELLVRSPGVMKEYWGNPDKTAEAIKDGWLCTGDLVSRDNDGYLAIKGRKKQMIIRGGENIYPIEIENVLYAHPDILEAAIAGVPDRIMGEEVKAVIVLKEGVSMDAAQLQEFLKTRLADYKIPKYISFVRELPRNPAGKVQVDKLVNL

SEQ ID NO: 10 - protein, 504 aa, synthetic construct
  REGION 1..504: note = Deltaproteobacteria bacterium
MNINIATWLTRNATIFANKTAIIDDRLKFTYKELDERVRRLATAFEAKGVKKGDRIAVLLPNNIEVVEALFAAARLGAIFVPLNWRLGIDELEYILNDCQPSLLIYKDDWQDRVDALNERLSSPPASIATAEATENPYEETIASNEPHTNDDYPACDGDDLMIIYTSGTTGNPKGAVLTYTNVFFQTINGWSLGTSPDGIALVLLPLFHVGGLNGSVAPLLHIGATVILQAKFKPAHVLKTIEDEQVTGVLGVPTIFRMLSEQPEFETTDLSKCDVLLSGGAPLPESLIELYHERGLEFRQGYGLTEASPGVTGMGPGECRQKIGSAGRQILYTEVRIVNDAGKQMPTSESGEIIVRGPNVMRGYWNKPEATAKAIKNGWLHTGDVGYFDEDGFLFIVDRMKDMIISGGENIYPAEIEKLLAAHPKVQQATVVGRPDEKWGEIPVAVVVPSDKDLTKDELKAFLTDLARYKQPKAYHFFETLPLNASGKVVKAEVKKQLDALDK

SEQ ID NO: 11 - protein, 515 aa, synthetic construct
  REGION 1..515: note = Spongiibacter tropicus
MIWSIAELIQRQVSRGSKRPMLFYGEQTLSYGDIDAQADRVAAGLHAAGLSAGDRVALLDKNSPEQFEILFGNSKAGVVTVPINWRLSPDEVIYILNHSEARIVFVGAEFAEPLMQRRAKLEREVRIIVMDEDAGDAYSLWLTAAPTTAPRIETKPDDVSLQLYTSGTTGRPKGVMLTSRNLFTLLNASDDLWGLDSHSISIVCMPLFHIGGIGWALACLLHGGAVCLTREFEPKSMLATIEAHKATHANFVPAMLAALTDAAQHVEYDYSSLALILYGTAPISKPLLMASMRTFGCCFNQVYGLTETTSAITQLDWDDHKAGLDRLQSAGRAYPWIELKIVDPNSERQCRSGETGELWVKSAQNMKGYWRDPEATLRSQKPGGWLATGDAGYLDEDGYLFLTDRVKDMIVSGGENIYPAEVERVLVEHDMVNEVAVIGIPHDRWGEEVKAMVVPASDVRLDEAELIDWCREHLARYKCPKSVDFIDALPRNATGKVLKRILREPYWCEKDRAIN

(19) United States
(12) Patent Application Publication
(10) Pub. No.: US 2026/0035721 A1
(43) Pub. Date: Feb. 5, 2026

(54) ENZYMATIC SYNTHESIS OF AMIDE-CONTAINING MOLECULES

(71) Applicant: UT-Battelle, LLC, Oak Ridge, TN (US)

(72) Inventors: Joshua K. Michener, Oak Ridge, TN (US); Jeff C. Foster, Knoxville, TN (US); Liangyu Qian, Lenoir City, TN (US); Isaiah Dishner, Oak Ridge, TN (US)

(21) Appl. No.: 19/285,556

(22) Filed: Jul. 30, 2025

Related U.S. Application Data
(60) Provisional application No. 63/677,609, filed on Jul. 31, 2024.

(51) Int. Cl. C12P 13/02; C07C 237/04; C08G 69/02; C12N 9/00

(52) U.S. Cl. CPC C12P 13/02; C07C 237/04; C08G 69/02; C12N 9/93; C12Y 603/01

(57) ABSTRACT

A method for producing amide-containing molecules of the formula:

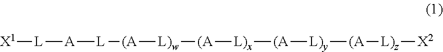

wherein: X¹ and X² are independently selected from NH₂ and COOH; L is independently, in each instance, selected from hydrocarbon linkers containing 1-12 carbon atoms optionally containing one or more heteroatoms selected from O, N, S, and halogen atoms, and are the same or different; A independently represents an —NHC(O)— or —C(O)NH— linkage; w, x, y, and z are independently 0 or 1; the method comprising reacting at least two reactant molecules that form an amide bond with each other via X¹ and X² groups, in the presence of an amide synthetase enzyme, wherein the at least two reactant molecules are independently selected from the following formulas:

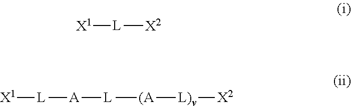

wherein v is 0, 1, or 2.

### CROSS REFERENCE TO RELATED APPLICATION

The present application claims benefit of U.S. Provisional Application No. 63/677,609, filed on Jul. 31, 2024, all of the contents of which are incorporated herein by reference.

### STATEMENT REGARDING FEDERALLY SPONSORED RESEARCH

This invention was made with government support under Prime Contract No. DE-AC05-00OR22725 awarded by the U.S. Department of Energy. The government has certain rights in the invention.

### INCORPORATION BY REFERENCE OF SEQUENCE LISTING

The Sequence Listing in XML file, named as 44935_5621_1_SequenceListing of 20,480 bytes, created on Jul. 10, 2025, and submitted to the United States Patent and Trademark Office via Patent Center, is incorporated herein by reference.

### FIELD OF THE DISCLOSURE

The present disclosure generally relates to methods of producing amide-containing molecules and amide polymers produced therefrom. The present disclosure more particularly relates to enzymatic methods of producing amide-containing molecules, and more particularly, wherein the enzyme is an amide synthetase enzyme.

### BACKGROUND

Polyamides are polymers linked by amide groups and synthesized by the reaction of diamines with dicarboxylic acids, by the condensation of ω-amino acids, or by ring-opening polymerization of cyclic lactams. Nylons, a prominent category of synthetic thermoplastic polyamides, were produced at an estimated scale of 9 million tons in 2020 (Global Industry Analysts, 2020). Applications of nylons are widespread due to their exceptional mechanical strength, thermal stability and chemical resistance, making them indispensable in industries such as textiles, automotive manufacturing, and additive manufacturing. The continued demand for high-performance materials has driven research efforts aimed at improving the synthesis and functional properties of nylons, with an emphasis on achieving higher molecular weight and expanding the scope of usable precursors.

Commercial nylons are typically produced by polymerizing either a single ω-amino acid (e.g., nylon 6, also known as PA6) or a stoichiometric nylon salt formed from a diamine and a diacid (e.g., nylon-66, or PA66). Production of these commercial polyamides is generally complex and energy intensive. For example, production of renewable nylon-6,6 requires separately synthesizing and purifying the two starting materials (adipic acid and hexamethylene diamine), co-crystalizing them into a salt, and polymerizing the salt. Less complex and energy efficient methods for producing these common nylons would substantially advance the nylon industry.

There is also interest in polyamides based on very different monomer units than currently available. However, the available methods are generally cumbersome and difficult to scale up. In particular, efforts to improve nylon sustainability have led to interest in incorporating bioderived components, such as glutarate and cadaverine. However, polymerization of nylon salts containing glutarate often yields polymers with low molecular weight, which has hindered their broader exploration and application. Despite the limited studies on glutarate-based nylons, nylon 5,5 composed of cadaverine and glutarate, has been reported to possess desirable properties, including a high melting temperature and excellent thermal stability (L. Zhou et al., European Polymer Journal, 180, 111618, Nov 2022). Nylon 6,5 remains largely unexplored but shows a distinct hydrogen bonding pattern and moderate crystallinity (E. Navarro et al., Macromolecules, 28, 8742-8750, 1995; C. M. Rohles et al., Green Chemistry, 20, 4662-4674, 2018). Although these less common polyamides are of interest, the conventional methods used for producing them are complex and economically infeasible. Efforts in finding more flexible and cost-effective synthetic routes that can more readily incorporate non-conventional monomers to expand the scope of polyamides have been largely unsuccessful.

Moreover, commercial polyamides represent only a small fraction of the potential diversity in polyamide sequences. Conventional polymers are composed of one (e.g. Nylon 6) or two (e.g. Nylon 66) monomers arranged in a repeating sequence. Polymers with more complicated sequences could have improved or unusual properties, as evidenced by proteins. However, copolymerization of mixed monomers gives a random arrangement of monomers and poor material properties. The relative simplicity of current nylon sequences derives from the challenges of controlling the incorporation of complex monomer mixtures during polymerization. New methods for controlling polymer sequences could produce new polyamides with special or improved properties, but such methods have remained largely elusive.

Thus, there remains a need for producing both simple and complex amide-containing molecules by more straightforward and cost-effective means than currently available. There would be an added advantage if such a method could be capable of producing a diverse range of amide-containing molecules varying in complexity and/or size. There would be a further advantage if such a method could be easily scaled up on an industrial level to meet large scale demands. Such a method would represent a significant advance in the art of producing polyamide polymers since the method would be capable of producing numerous types of building block amide molecules that could be used to produce a corresponding diverse array of polyamide polymers by facile and cost effective means.

### SUMMARY OF THE DISCLOSURE

The present disclosure is foremost directed to a novel enzymatic-based method that can be used for producing a wide range of amide-containing molecules of varying complexity and size. The amide-containing molecules may be used as building blocks (i.e., “monomers”) for producing amide polymers of corresponding complexity. The method may be used, for example, to produce novel sequenced oligoamides (e.g., amide diads, triads, tetrads, or higher) which can be used as building blocks for producing correspondingly novel amide polymers. The amide-containing molecules may find other uses, e.g., telechelics, waterborne coatings, elastomers, formulation additives, phase-change materials, or side-chain motifs. The method for producing the amide-containing molecules is advantageously straightforward and cost-effective. The method is further advantageous by being amenable for large-scale industrial production.

More particularly, the method is used for producing amide-containing molecules of the formula:

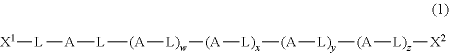

wherein: X¹ and X² are independently selected from NH₂ and COOH; L is independently, in each instance, selected from hydrocarbon linkers containing 1-12 carbon atoms optionally containing one or more heteroatoms selected from O, N, S, and halogen atoms, and are the same or different; A independently represents in each instance an —NHC(O)— or —C(O)NH— linkage; w, x, y, and z are independently 0 or 1;
the method comprising reacting at least two reactant molecules that form an amide bond with each other via X¹ and X² groups, in the presence of an amide synthetase enzyme (e.g., DdaG, SfaB, DesD, AsbA, AsbB, AcsA, or a homolog of any of these), wherein the at least two reactant molecules are independently selected from the following formulas:

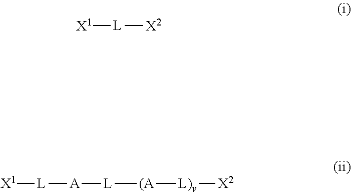

wherein v is 0, 1, or 2 and the at least two reactant molecules undergoing the reaction are selected from Formula (i), or from Formula (ii), or from at least one molecule of Formula (i) and at least one molecule of Formula (ii); wherein the reaction of the at least two reactant molecules above results in an amide-containing molecule of Formula (1).

In some embodiments, at least one L in Formula (1) is selected from linear or branched alkylene linkers containing 1-12 carbon atoms and optionally one or more heteroatoms selected from O, N, S, and halogen atoms. In other embodiments, at least one L in Formula (1) includes a ring and optionally contains one or more heteroatoms selected from O, N, S, and halogen atoms. In some embodiments, the at least one reactant molecule of Formula (i) is selected from adipic acid (A), hexamethylenediamine (M), succinic acid (S), glutaric acid (G), pimelic acid (P), suberic acid (SU), azelaic acid (Z), sebacic acid (SE), muconic acid (MU), p-xylylenediamine (X), terephthalic acid (T), cadaverine (C), cis-1,4-cyclohexanediamine (N), 4-aminobutyrate (B), 5-aminovalerate (B), 6-aminohexanoic acid (H), 4-amino-2-hydroxybutanoic acid (E), and 4-amino-3-hydroxybutanoic acid (F), wherein 6-aminohexanoic acid is known to be synonymous with 6-aminocaproic acid. In some embodiments, any two, three, or four of the above reactant molecules (provided they can form an amide bond with each other) may be reacted to produce a diad, triad, or tetrad, respectively, and wherein a reactant molecule of Formula (ii) may or may not be present for further reaction to produce a higher oligoamide molecule.

In particular embodiments, the reaction proceeds by reacting two reactant molecules of Formula (i) that form an amide bond with each other in the presence of the amide synthetase enzyme to result in a dimeric (diad) amide-containing molecule of the following formula:

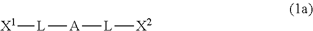

wherein X¹, X², L, and A are as defined above. In some embodiments, the two reactant molecules of Formula (i) are selected from adipic acid (A), hexamethylenediamine (M), succinic acid(S), glutaric acid (G), pimelic acid (P), suberic acid (SU), azelaic acid (Z), sebacic acid (SE), muconic acid (MU), p-xylylenediamine (X), terephthalic acid (T), cadaverine (C), cis-1,4-cyclohexanediamine (N), 4-aminobutyrate (B), 5-aminovalerate (B), 6-aminohexanoic acid (H), 4-amino-2-hydroxybutanoic acid (E), and 4-amino-3-hydroxybutanoic acid (F). In other embodiments, the two reactant molecules may be more generally selected from a diamine (or more particularly, an α,ω-alkylenediamine (e.g., M or C)) and a dicarboxylic acid (or more particularly, an α,ω-alkylenedicarboxylic acid (e.g., A, S, G, P, SU, Z, or SE)), wherein the diamine and dicarboxylic acid react with each other to form a dimeric amide molecule (i.e., amide dimer). For producing dimeric amide- containing molecules within the scope of Formula (1a), particularly where the linkers (L) do not contain a ring, the amide synthetase enzyme may more particularly be a homolog of SfaB, such as AS9, AS17, AS41, AS3, or AS8. For producing dimeric amide-containing molecules within the scope of Formula (1a), particularly wherein at least one linker (L) contains a ring (e.g., phenylene), the amide synthetase enzyme may more particularly be any one of SfaB, DesD, AcsA, or homologs thereof, or more particularly AcsAor a homolog thereof. The amide dimer may be further reacted with any one or more of the above reactant molecules using the same or different amide synthetase enzyme to produce an amide trimer, tetramer, pentamer, or hexamer within the scope of Formula (1).

In a first set of embodiments, the reaction proceeds by reacting three or more molecules of Formula (i) that form an amide bond with each other in the presence of an amide synthetase enzyme to result in a trimeric (triad) or higher oligomeric amide- containing molecule of the Formula (1) wherein w is 1. The reaction may be conducted as a single-step or multi-step reaction, wherein a multi-step reaction employs the same or different enzyme for each step.

In a second set of embodiments, the reaction proceeds by reacting at least one molecule of Formula (i) with at least one molecule of Formula (ii) that form an amide bond with each other in the presence of an amide synthetase enzyme to result in a trimeric or higher oligomeric amide-containing molecule of the Formula (1) wherein w is 1. The reaction may be conducted as a single-step or multi-step reaction, wherein a multi-step reaction employs the same or different enzyme for each step.

In a third set of embodiments, the reaction proceeds by reacting at least one molecule of Formula (i) with at least one molecule of Formula (ii) that form an amide bond with each other in the presence of an amide synthetase enzyme to result in a tetrameric (tetrad) or higher oligomeric amide-containing molecule of the Formula (1) wherein w and x are each 1. The reaction may be conducted as a single-step or multi-step reaction, wherein a multi-step reaction employs the same or different enzyme for each step.

In a fourth set of embodiments, the reaction proceeds by reacting two or more molecules of Formula (ii) that form an amide bond with each other in the presence of an amide synthetase enzyme to result in a tetrameric or higher oligomeric amide-containing molecule of the Formula (1) wherein w and x are each 1. The reaction may be conducted as a single-step or multi-step reaction, wherein a multi-step reaction employs the same or different enzyme for each step. In some embodiments, the amide synthetase enzyme is or includes a DesD homolog.

In another aspect, the present disclosure is directed to the resulting amide-containing molecules produced by the method described above. In some embodiments, the amide-containing molecules have the following formula: X¹-L-A-L-X² (1a). In other embodiments, the amide-containing molecules have the following formula:

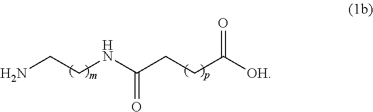

In other embodiments, the amide-containing molecules have the following formula:

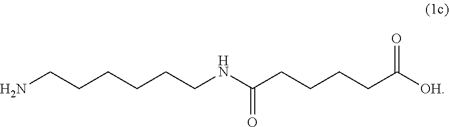 [SMILES: NCCCCCCNC(=O)CCCCC(=O)O]

In other embodiments, the amide-containing molecules have the following formula:

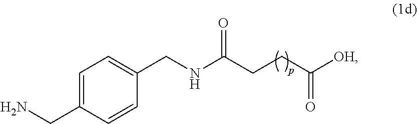

wherein p is an integer in a range of 1-11. In particular embodiments, p is an integer in a range of 1-5.

In another aspect, the present disclosure is directed to amide polymers produced from any of the amide-containing molecules described above within the scope of Formula (1). In more particular embodiments, the amide polymer is a polymerized version of any of Formulas (1a), (1b), (1c), or (1d). As the amide polymers contain at least two different units, the amide polymers are understood to be copolymers. The amide copolymers may have any of the arrangements well known in the art, such as alternating, periodic, random, or block arrangements, or a combination of such arrangements. The amide copolymer may or may not also include non-amide units or blocks thereof (e.g., by reaction of the amide molecule with epoxy or isocyanate molecules).

The present disclosure demonstrates that chemically-synthesized nylon diads can be used to generate a range of polyamides with higher molecular weights compared to polymerization of traditional salts. The above result was achieved using a simplified biosynthetic strategy in which amide synthetases were used to produce nylon diad precursors from unprotected bifunctional substrates. By coupling amide bond formation with an ATP-regeneration system, the biocatalytic process was optimized and scaled up, thus establishing it as a viable method for synthesizing nylon diads. It has herein been further shown that amide synthetases exhibit a broad substrate scope and can catalyze the assembly of a diverse range of nylon-relevant diacids, diamines, and ω-amino acids. This approach provides a facile route for the polymerization of challenging monomers and expands the potential for tailoring nylon properties through enzymatically derived precursors.

The present disclosure further demonstrates that synthesizing multi-component oligoamides, such as triads and tetrads, requires enzymes capable of ligating longer and more diverse substrates. NRPS-independent siderophore (NIS) synthetases, which catalyze ATP-dependent ligation of carboxylates with nucleophiles in siderophore biosynthesis, are promising candidates due to their ability to natively condense longer substrates and may also perform macro-cyclization. NIS synthetases are classified into three types based on substrate specificity: Type A (citrate), Type B (α-ketoglutarate), and Type C (citrate or succinate derivatives). These enzymes exhibit low sequence homology (20-30%), reflecting their diverse substrate preferences. However, their potential for polymer-relevant monomer ligation remained unexplored.

The present disclosure further demonstrates that NIS synthetases are active on polymer-relevant substrates and provide unique activities compared to previously characterized enzymes. Notably, DesD, a Type C NIS synthetase, proved useful in synthesizing a diverse number of oligoamides. Using an enzyme cascade involving two or three enzymes, an ω-amino acid triad and tetrad were synthesized by a one-pot reaction or sequential reaction directly from unprotected substrates. Furthermore, DesD and DdaG were herein found to catalyze regioselective ligation of asymmetric substrates, providing the basis of sequence control over amide bond formation. The presently described method not only provides a viable method for enzymatically synthesizing multi-component nylon oligomers, but also facilitates the characterization of polyamide sequence-function relationships and the design of new polymers.

### BRIEF DESCRIPTION OF THE DRAWINGS

FIG. 1. Data showing that AS9 (SfaB homolog) can ligate adipic acid (A) and hexamethylenediamine (M) into PA66 monomer (MA). In vitro biochemical assays were performed by incubating adipic acid with hexamethylenediamine in the presence of SfaB or a no-enzyme control. Reactions were carried out in duplicate in 50 μL volumes containing 100 mM HEPES (pH 8.0), 10 mM ATP, 10 mM MgCl₂, 10 μM enzyme or control buffer, 5 mM adipic acid and 5 mM hexamethylenediamine. The reactions were incubated at 30° C. for 16 h with shaking at 200 rpm and quenched by the addition of 50 μL methanol. After centrifugation at 4,000 rpm for 10 min, the supernatants were analyzed by I.DOT/OPSI-MS. Product formation was quantified by comparison to chemically synthesized standards.

FIG. 2. Data showing that AcsA can ligate adipic acid (A) and p-xylylenediamine (X) into XA. In vitro biochemical assays were performed by incubating adipic acid with hexamethylenediamine in the presence of AcsA or a no-enzyme control. Reactions were carried out in triplicate in 50 μL volumes containing 100 mM HEPES (pH 8.0), 10 mM ATP, 10 mM MgCl₂, 10 μM enzyme or control buffer, 5 mM adipic acid and 5 mM p-xylylenediamine. The reactions were incubated at 30° C. for 16 h with shaking at 200 rpm and quenched by the addition of 50 μL methanol. After centrifugation at 4,000 rpm for 10 min, the supernatants were analyzed by I.DOT/OPSI-MS. The structure was confirmed by analysis of fragmentation patterns by MS².

FIG. 3. Data showing that SfaB can ligate PA66 monomer (MA) with adipic acid (A) into diacid triad (AMA). In vitro biochemical assays were performed by incubating adipic acid with PA66 monomer in the presence of SfaB or a no-enzyme control. Reactions were carried out in triplicate in 50 μL volumes containing 100 mM HEPES (pH 8.0), 10 mM ATP, 10 mM MgCl₂, 10 μM enzyme or control buffer, 5 mM adipic acid and 5 mM synthesized PA66 monomer standard. The reactions were incubated at 30° C. for 16 h with shaking at 200 rpm and quenched by the addition of 50 μL methanol. After centrifugation at 4,000 rpm for 10 min, the supernatants were analyzed by I.DOT/OPSI-MS. The structure was confirmed by analysis of fragmentation patterns by MS².

FIGS. 4A-4B. FIG. 4A is a scheme showing the regioselective ligation of PA66 (MA) monomer with 6-aminocaproic acid (H) as facilitated by DesD enzyme to produce PA666 monomer. FIG. 4B presents data showing that DesD can regioselectively ligate PA66 monomer (MA) with 6-amino caproic acid (H) into MAH. In vitro biochemical assays were performed by incubating PA66 monomer with 6-amino caproic acid in the presence of DesD or a no-enzyme control. Reactions were carried out in triplicate in 50 μL volumes containing 100 mM HEPES (pH 8.0), 10 mM ATP, 10 mM MgCl₂, 10 μM enzyme or control buffer, 5 mM PA66 monomer and 5 mM 6-amino caproic acid. The reactions were incubated at 30° C. for 16 h with shaking at 200 rpm and quenched by the addition of 50 μL methanol. After centrifugation at 4,000 rpm for 10 min, the supernatants were analyzed by I.DOT/OPSI-MS. Both possible triad standards, MAH and HMA, were chemically synthesized, and MS² was used to identify their unique fragment ions. The MS²spectrum of the enzymatic product matched only the MAH standard, indicating that DesD selectively forms a single ω-amino acid triad product.

FIGS. 5A-5B. FIG. 5A is a scheme showing the regioselective ligation of PA66 monomer (MA) with PA6 dimer (HH) as facilitated by DesD enzyme to produce a HHMA monomer. FIG. 5B presents data showing that DesD can regioselectively ligate PA66 monomer (MA) with PA6 dimer (HH) into HHMA. In vitro biochemical assays were performed by incubating PA66 monomer with PA6 dimer in the presence of DesD or a no-enzyme control. Reactions were carried out in triplicate in 50 μL volumes containing 100 mM HEPES (pH 8.0), 10 mM ATP, 10 mM MgCl₂, 10 μM enzyme or control buffer, 5 mM PA66 monomer and 5 mM PA6 dimer. The reactions were incubated at 30° C. for 16 h with shaking at 200 rpm and quenched by the addition of 50 μL methanol. After centrifugation at 4,000 rpm for 10 min, the supernatants were analyzed by I.DOT/OPSI-MS. The structure was confirmed by analysis of fragmentation patterns by MS².

FIGS. 6A-6B. FIG. 6A presents data showing that an enzyme cascade including DdaG and DesD can ligate succinic acid (S), hexamethylenediamine (M) and 8-aminooctanoic acid (R) into SMR. A simultaneous one-pot reaction was carried at 100 μL containing 100 mM HEPES (pH 8.0), 40 mM ATP, 40 mM MgC₁₂, 10 μM DdaG, 20 μM DesD, 5 mM succinic acid, 5 mM hexamethylenediamine, and 5 mM 1,8-octanoic amino acid and was incubated at 30° C. for 16 h with shaking at 200 rpm. The reaction was quenched by adding 100 μL methanol. The samples were centrifuged at 4,000 rpm for 10 min, and the supernatant was subjected to MS analysis to determine the formation of corresponding products. FIG. 6B is a scheme showing the enzyme cascade process for producing SMR as facilitated by DdaG and DesD.

FIGS. 7A-7B. FIG. 7A presents data showing that an enzyme cascade including DdaG, SfaB and DesD can ligate succinic acid(S), hexamethylenediamine (M), 1,8-diaminooctane (O) and glutaric acid (G) into SMGO or MGOS. A one-pot sequential reaction strategy was developed. First, DdaG was used to ligate S and M to form the MS diad. 100 μL reactions containing 100 mM HEPES (pH 8.0), 10 mM ATP, 10 mM MgCl₂, 10 μM DdaG, 5 mM disodium succinate, and 5 mM hexamethylenediamine were incubated at 30° C. for 24 h with shaking at 200 rpm to generate the MS diad. After 24 h, reactions were heat-inactivated at 95° C. for 5 min, followed by centrifugation at 4,000 rpm for 10 min. DesD (20 μM final), 5 mM 1,8-diaminooctane, 20 mM ATP, and 10 mM MgCl₂ were added into the supernatant, and the reaction was incubated for another 24 h under identical conditions to generate the MSO triad. After a further 24 h, the reaction was again heat-inactivated and centrifuged. Then the supernatant was supplemented with 10 μM SfaB, 5 mM glutaric acid, 20 mM ATP, and 10 mM MgCl₂ to generate the tetrad MSOG or GMSO. The reaction proceeded for an additional 24 h under identical conditions and was quenched and centrifuged as before, and the supernatant was collected for final I.DOT/OPSI-MS analysis. In addition to the desired product, a range of side products was observed to form, including MS, MG, OS, OG, and SMG, likely due to excess substrate and limited substrate specificity. FIG. 7B is a scheme showing the enzyme cascade process for producing SMGO or MGOS as facilitated by DdaG and DesD.

### DETAILED DESCRIPTION

In a first aspect, the present disclosure is directed to a method of producing amide-containing molecules of the formula:

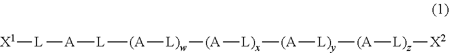

The variables X¹ and X² in Formula (1) are independently selected from NH₂ and COOH. In some embodiments, X¹ and X² are both NH₂. In other embodiments, X¹ and X² are both COOH. In other embodiments, X¹ is NH₂ and X² is COOH, or X¹ is COOH and X² is NH₂.

The variable A independently represents in each instance an —NHC(O)— or —C(O)NH— linkage. As shown in Formula (1), each instance of A functions as a linkage between two L linkers.

The variables w, x, y, and z are independently 0 or 1, wherein 0 indicates absence of the subtended variable and 1 indicates presence of the subtended variable. Moreover, if w is 0, then x, y, and z are 0, and if x is 0, then y and z are 0, and if y is 0, then z is 0. In the case where w, x, y, and z are 0, the Formula (1) reduces to X¹-L-A-L-X² (1a′), which is herein referred to as a dimeric amide-containing molecule (or “diad”). In the case where w is 1 and x, y, and z are 0, the Formula (1) reduces to X¹-L-A-L-(A-L)-X² (1b′), which is herein referred to as a trimeric amide-containing molecule (or “triad”). In the case where w and x are 1 and y and z are 0, the Formula (1) reduces to X¹-L-A-L-(A-L)-(A-L)-X² (1c′), which is herein referred to as a tetrameric amide-containing molecule (or “tetrad”). In the case where w, x, and y are 1 and z is 0, the Formula (1) reduces to X¹-L-A-L-(A-L)-(A-L)-(A-L)-X² (1d′), which is herein referred to as a pentameric amide-containing molecule (or “pentad”). In the case where w, x, y, and z are 1, the Formula (1) reduces to X¹-L-A-L-(A-L)-(A-L)-(A-L)-(A-L)-X² (1e′), which is herein referred to as a hexameric amide- containing molecule (or “hexad”).

The variable L in Formula (1) is independently, in each instance, selected from hydrocarbon linkers containing 1-12 carbon atoms optionally containing one or more heteroatoms selected from O, N, S, and halogen atoms (i.e., F, Cl, Br, or I). L may be the same or different in Formula (1). In some embodiments, L in all instances is the same. In other embodiments, at least two instances of L in Formula (1) are different. The term “hydrocarbon linker containing 1-12 carbon atoms” refers to any divalent (linking) group composed of at least (or solely) carbon and hydrogen atoms. The L linkers independently contain or do not contain (i.e., optionally contain), in each instance, one or more heteroatoms. The hydrocarbon linker may be linear, branched, cyclic, or may contain a combination of two or more of these features, such as a linear portion linked to a cyclic portion or a branched portion linked to a cyclic portion. The hydrocarbon linker may, in addition, be saturated or unsaturated. An unsaturated hydrocarbon linker contains at least one carbon-carbon double or triple bond and may be linear, branched, or cyclic.

In some embodiments, precisely or at least one or two L in Formula (1) is/are selected from linear or branched alkylene linkers containing 1-12 carbon atoms and optionally one or more heteroatoms selected from O, N, S, and halogen atoms, wherein “alkylene” is understood to be a divalent linker derived from an alkyl (unsaturated) group containing 1-12 carbon atoms. In the case wherein at least one heteroatom is present, the heteroatom may be, for example, an ether (—O—) linkage connecting two carbon atoms, hydroxy group (OH) or thio group (SH) substituting a hydrogen atom, amine (—NH—) linkage connecting two carbon atoms, an oxygen atom substituting two hydrogen atoms on one carbon atom to form a carbonyl (C═O), or a halogen atom (e.g., F, Cl, or Br) substituting a hydrogen atom. Some examples of alkylene linkers include linear types, such as methylene (—CH₂—), ethylene/dimethylene (—CH₂CH₂—), n-propylene/trimethylene (—CH₂CH₂CH₂—), tetramethylene, pentamethylene, hexamethylene, and the like, and branched types, such as isopropylene (—CH(CH₃)CH₂—) or isobutylene (—CH(CH₂CH₃)CH₂— or —CH(CH₃)CH(CH₃)—). In other embodiments, precisely or at least one or two L in Formula (1) includes a ring and optionally contains one or more heteroatoms selected from O, N, S, and halogen atoms. In other embodiments, precisely or at least one or two L in Formula (1) is/are selected from linear or branched alkylene linkers containing 1-12 carbon atoms and optionally one or more heteroatoms selected from O, N, S, and halogen atoms, and precisely or at least one or two L in Formula (1) includes a ring and optionally contains one or more heteroatoms selected from O, N, S, and halogen atoms.

In some embodiments, at least one or all of the hydrocarbon linkers (L) have the following formula: —(CH₂)r— wherein r is an integer of 1-12 or an integer within a narrower range thereof, e.g., 1-8, 1-6, 1-5, 1-4, 1-3, 2-12, 2-8, 2-6, 2-5, 2-4, 3-12, 3-8, 3-6, 4-12, 4-8, 4-6, 5-12, 5-8, 6-12, or 6-8. In some embodiments, all L variables in Formula (1) have the formula —(CH₂)r—, are the same or different, and independently select r to be in any one of the ranges recited above. In some embodiments, the formula —(CH₂)r— can (optionally) include a level of unsaturation by removing two hydrogen atoms on connecting carbon atoms and replacing them with a double bond connecting the carbon atoms. Some examples of unsaturated versions of —(CH₂)r— include —CH₂—CH═CH—, —CH₂—CH═CH—CH₂—, —CH═CH—CH═CH—, —CH₂—CH═CH—CH—CH—CH₂—, —CH₂—CH═CH—CH—CH—, and —CH₂—CH═CH—CH₂—CH═CH—. One or more heteroatoms, if present, may insert between carbon atoms, such as in —CH₂—CH₂—O—CH₂—CH₂—, —CH₂—CH₂—S—CH₂—CH₂—, or —CH₂—CH₂—NH—CH₂—CH₂—, or the one or more heteroatoms may function as a substituent, such as in —CH₂—CH₂—CH(OH)—CH₂—CH₂— or —CH₂—CH₂—CH(X)—CH₂—CH₂— where X is a halogen atom, or the one or more heteroatoms may otherwise be bound to a carbon atom, such as in —CH₂—CH₂—C(═O)—CH₂—CH₂—. Heteroatoms in L, if present, do not form an amide linkage since the presence of such linkages is covered by variable A. However, one or more other possible heteroatom-containing linkages may (or may not) be present in one or more L, such as one or more ester, urethane, carbonate, urea, or azo linkages. In some embodiments, the formula —(CH₂)r— can (optionally) include branching by substituting one or more H atoms in the formula with a methyl, ethyl, n-propyl or isopropyl group, provided that the maximum number of carbon atoms in —(CH₂)r— is 12. Different L variables in Formula (1) may be independently selected from any of the r values or ranges thereof, levels of unsaturation, presence or absence of heteroatoms, and presence or absence of branching.

In other embodiments, at least one or all of the hydrocarbon linkers (L) contain a linking cyclic group (i.e., is a cyclic-containing linker), wherein the linking cyclic group is composed of at least carbon and hydrogen atoms and may optionally contain one or more heteroatoms. The linking cyclic group may be, for example, phenylene, cyclopentadienylene, cyclohexylene, cyclopentylene, furanylene, pyridinylene, or imidazolylene (which indicate divalent forms of benzene, cyclopentadiene, cyclohexane, cyclopentane, furan, pyridine, and imidazole rings, respectively). In some embodiments, at least one L is a linking cyclic group, i.e., of the formula -X- wherein X represents a linking cyclic group, such as any of those provided above. In other embodiments, at least one L includes a linking cyclic group inserted between carbon atoms within the linkage —(CH₂)r—, such as in a linker of the formula —CH₂-X-CH₂— or —CH₂CH₂-X-CH₂CH₂—, wherein X represents a linking cyclic group, such as any of those provided above, provided that the maximum number of carbon atoms in the cyclic-containing linker is 12.

In some embodiments, one or more L in Formula (1) are selected from linkers of the formula —(CH₂)r— and one or more L are selected from cyclic-containing linkers described above. In more specific embodiments, one or more L are selected from linkers of the formula —(CH₂)r— and one or more L are selected from cyclic-containing linkers of the formula —CH₂-X-CH₂— or —CH₂CH₂-X-CH₂CH₂—, wherein X represents a cyclic linking group, such as any of those provided above.

In the method, precisely or at least two reactant molecules that form an amide bond with each other are reacted with each other in the presence of an amide synthetase enzyme to result in an amide-containing molecule within the scope of Formula (1). In some embodiments, precisely or at least three or four reactant molecules that form an amide bond with each other are reacted with each other in the presence of an amide synthetase enzyme to result in an amide-containing molecule within the scope of Formula (1).

The at least two, three, or four reactant molecules are independently selected from the following formulas:

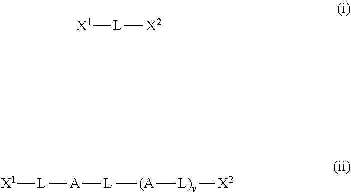

In the above Formulas (i) and (ii), v is 0, 1, or 2, and X¹, X², L, and A are defined according to any of the definitions and examples provided above under Formula (1). When v is 0, the reactant molecule according to Formula (ii) is a dimeric amide molecule. When v is 1, the reactant molecule according to Formula (ii) is a trimeric amide molecule. When v is 2, the reactant molecule according to Formula (ii) is a tetrameric amide molecule.

In a first set of embodiments, precisely or at least two, three, or four reactant molecules undergoing the reaction are selected from Formula (i). In a second set of embodiments, precisely or at least two, three, or four reactant molecules undergoing the reaction are selected from Formula (ii). In a third set of embodiments, precisely or at least two, three, or four reactant molecules undergoing the reaction are selected from precisely or at least one or two molecules of Formula (i) and precisely or at least one or two molecules of Formula (ii).

The enzyme that catalyzes amide bond formation is an amide synthetase enzyme, some of which are known as NIS synthetases. These enzymes use ATP to activate a carboxylic acid and condense that activated intermediate with an amine to form an amide bond. As well known, NIS synthetases are classified into three types based on substrate specificity: Type A (citrate), Type B (α-ketoglutarate), and Type C (citrate or succinate derivatives). Amide synthetase enzymes exhibit low sequence homology (20-30%), reflecting their diverse substrate preferences. As further discussed below, it is here shown that these enzymes accept non-native substrates such as succinic/glutaric/adipic acids and diamines such as cadaverine and hexamethylene diamine to form diad and triad products. These products can then be polymerized to yield new polymers with defined sequences. In some embodiments, the amide synthetase enzyme is selected from any one of DdaG, SfaB, DesD, AsbA, AsbB, AcsA, and homologs thereof.

As used herein, the term “homologs” is known in the art and refers to related amide synthetase molecules that may share a common ancestor and are similar in sequence (i.e., having substantial homology in sequences) and function (i.e., having similar substrate preference and producing similar products). Homology between sequences can be determined using software programs readily available in the art, such as those discussed in Current Protocols in Molecular Biology (F. M. Ausubel et al., eds., 1987) Supplement 30, section 7.718, Table 7.71. Examples of alignment programs include but are not limited to: Mac Vector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters. Sequences having “substantial homology” include sequences that are at least 45% identical, at least 55% identical, at least 65% identical, at least 75% identical, at least 85% identical, or at least 95% identical. Sequences having “substantial homology” also include sequences that are at least 98% or 99% identical, or that are 100% identical. Sequences having “substantial homology” also encompasses variants that differ by not more than 4 or 5 amino acids, or not more than 2 or 3 amino acids. Sequences having “substantial homology” also encompasses variants that differ by only 1 amino acid. Sequences having “substantial homology” also encompasses variants that have differences in amino acids as a result of polymorphism.

### Exemplary Protein Sequences of Amide Synthetases

| SEQ ID NO | Name and Species |
|---|---|
| 1 | DdaG from Pantoea agglomerans |
| 2 | SfaB from Streptomyces thioluteus |
| 3 | AsbA from Bacillus thuringiensis |
| 4 | AsbB from Bacillus thuringiensis serovar monterrey |
| | (BGSC 4AJ1) |
| 5 | AcsA from Pseudomonas fluorescens |
| 6 | DesD from Streptomyces coelicolor A3(2) |
| 7 | AS9 from Actinomycetales bacterium mxb001 |
| 8 | AS41 from Lysinibacillus sp. |
| 9 | AS17 from Smithella sp. PtaU1.Bin162 |
| 10 | AS8 from Deltaproteobacteria bacterium |
| 11 | AS3 from Spongiibacter tropicus |

In some embodiments, the amide synthetase enzyme is DdaG or a homolog thereof. The DdaG enzyme and/or homologs thereof have herein been found to be particularly effective in reacting aliphatic diamines (e.g., alkylenediamines) with aliphatic dicarboxylic acids (e.g., alkylenediacids), within the scope of Formula (i) or (ii), to produce dimeric or higher amide-containing molecules of Formula (1), (1a), or (1b), or more particularly PA54, PA64, or PA65 within the scope of Formula (1b). In particular embodiments, the aliphatic diacid is succinic acid. DdaG has herein been shown to be particularly effective in ligating succinic acid with one or more other reactant molecules within the scope of Formula (i) or (ii) to produce a dimeric or higher amide-containing molecule of Formula (1), (1a), or (1b). In some embodiments, DdaG comprises an amino acid sequence that is set forth in SEQ ID NO: 1. In some embodiments, DdaG or a homolog thereof comprises an amino acid sequence that is at least 85% or at least 95% identical to SEQ ID NO: 1.

In some embodiments, the amide synthetase enzyme is SfaB or a homolog thereof. The homolog of SfaB may be selected from, for example, AS9, AS17, AS41, AS3, or AS8, or a homolog of any of these. The SfaB enzyme and homologs thereof have herein been found to be particularly effective in reacting aliphatic diamines (e.g., alkylenediamines) with aliphatic dicarboxylic acids (e.g., alkylencdiacids) to produce dimeric or higher amide-containing molecules of Formula (1), (1a), or (1b), or more particularly PA66, PA56, PA65, PA55, PA64, PA54, or PAX6 within the scope of Formula (1b), or more particularly, PA66 of Formula (1c). In particular embodiments, the aliphatic diacid is an unsaturated aliphatic diacid (e.g., muconic acid) or a beta-keto acid (e.g., beta-ketoadipic acid). SfaB has herein been shown to be particularly effective in ligating muconic acid or a beta-keto acid (e.g., beta-ketoadipic acid) with one or more other reactant molecules within the scope of Formula (i) or (ii) to produce a dimeric or higher amide-containing molecule of Formula (1), (1a), or (1b). In some embodiments, SfaB comprises an amino acid sequence that is set forth in SEQ ID NO: 2. In some embodiments, SfaB or a homolog thereof comprises an amino acid sequence that is at least 85% or at least 95% identical to SEQ ID NO: 2. In some embodiments, an SfaB homolog selected from AS9, AS41, AS17, AS8, AS3, or homologs thereof. In some embodiments, AS9 comprises the amino acid sequence as set forth in SEQ ID NO: 7. In some embodiments, AS41 or a homolog thereof comprises an amino acid sequence that is at least 85% or at least 95% identical to SEQ ID NO: 7. In some embodiments, AS41 comprises the amino acid sequence as set forth in SEQ ID NO: 8. In some embodiments, AS41 or a homolog thereof comprises an amino acid sequence that is at least 85% or at least 95% identical to SEQ ID NO: 8. In some embodiments, AS17 comprises the amino acid sequence as set forth in SEQ ID NO: 9. In some embodiments, AS17 or a homolog thereof comprises an amino acid sequence that is at least 85% or at least 95% identical to SEQ ID NO: 9. In some embodiments, AS8 comprises the amino acid sequence as set forth in SEQ ID NO: 10. In some embodiments, AS8 or a homolog thereof comprises an amino acid sequence that is at least 85% or at least 95% identical to SEQ ID NO: 10. In some embodiments, AS3 comprises the amino acid sequence as set forth in SEQ ID NO: 11. In some embodiments, AS3 or a homolog thereof comprises an amino acid sequence that is at least 85% or at least 95% identical to SEQ ID NO: 11.

In some embodiments, the amide synthetase enzyme is DesD or a homolog thereof. The DesD enzyme and/or homologs thereof have herein been found to be particularly effective in reacting aryldiamines (e.g., p-xylylenediamine) with alkylenediacids to produce dimeric amide-containing molecules of Formula (1d). The DesD enzyme and/or homologs thereof have herein also been found to be particularly effective in reacting long-chain (e.g., C6, C7, C8, C9, C10, C11, or C12) alkylenediamines with long-chain alkylenediacids. The DesD enzyme and/or homologs thereof have herein also been found to be particularly effective in dimerizing N-succinylcadaverine. The DesD enzyme and/or homologs thereof have herein also been found to be particularly effective in reacting the PA66 monomer of Formula (1c) with an alkylenediamine, including those with an —NH— linkage (e.g., spermidine), to form a trimeric amide-containing molecule (i.e., triad). In some embodiments, DesD comprises an amino acid sequence that is set forth in SEQ ID NO: 6. In some embodiments, DesD or a homolog thereof comprises an amino acid sequence that is at least 85% or at least 95% identical to SEQ ID NO: 6.

In some embodiments, the amide synthetase enzyme is AsbA or a homolog thereof. The AsbA enzyme and/or homologs thereof have herein been found to be particularly effective in reacting a hydroxylated dicarboxylic acid or tricarboxylic acid (e.g., citrate) with an alkylenediamine, including those with an —NH— linkage (e.g., spermidine). In some embodiments, AsbA comprises an amino acid sequence that is set forth in SEQ ID NO: 3. In some embodiments, AsbA or a homolog thereof comprises an amino acid sequence that is at least 85% or at least 95% identical to SEQ ID NO: 3.

In some embodiments, the amide synthetase enzyme is AsbB or a homolog thereof. The AsbB enzyme and/or homologs thereof have herein been found to be particularly effective in reacting a hydroxylated dicarboxylic acid or tricarboxylic acid (e.g., citrate) with an alkylenediamine, including those with an —NH— linkage (e.g., spermidine). In some embodiments, AsbB comprises an amino acid sequence that is set forth in SEQ ID NO: 4. In some embodiments, AsbB or a homolog thereof comprises an amino acid sequence that is at least 85% or at least 95% identical to SEQ ID NO: 4.

In some embodiments, the amide synthetase enzyme is AcsA or a homolog thereof. The AcsA enzyme and/or homologs thereof have herein been found to be particularly effective in reacting aryldiamines (e.g., p-xylylenediamine) with alkylenediacids to produce dimeric amide-containing molecules of Formula (1d). The AcsA enzyme and/or homologs thereof have also herein been found to be particularly effective in reacting an alkylenediacid (e.g., adipic acid or azelaic acid) with an aryldiamine (e.g., p-xylylenediamine) or alkylenediamine (e.g., hexamethylenediamine). In some embodiments, AcsA comprises an amino acid sequence that is set forth in SEQ ID NO: 5. In some embodiments, AcsA or a homolog thereof comprises an amino acid sequence that is at least 85% or at least 95% identical to SEQ ID NO: 5.

In some embodiments, precisely or at least one or two reactant molecules of Formula (i) is/are selected from adipic acid (Ad), hexamethylenediamine (M), succinic acid (S), glutaric acid (G), pimelic acid (P), suberic acid (SU), azelaic acid (Z), sebacic acid (SE), muconic acid (MU), p-xylylenediamine (X), terephthalic acid (T), 1,4-cyclohexanedicarboxylic acid (CD), 2,5-furandicarboxylic acid (FD), cadaverine (C), 1,7-heptanediamine (HD), 1,8-octanediamine (O), 1,9-nonanediamine (ND), 1,10-decanediamine (DD), cis-1,4-cyclohexanediamine (N), 4-aminobutyrate (B), 5-aminovalerate (V), 6-aminohexanoic acid (H), 2-hydroxyglutaric acid (HG), α-ketoglutaric acid (K), 4-amino-2-hydroxybutanoic acid (E), and 4-amino-3-hydroxybutanoic acid (F), wherein the suffix “ate” is understood to include the neutral acid, e.g., “4-aminobutyrate” and “5-aminovalerate” are intended to include or be equivalent with “4-aminobutanoic acid” and “5-aminovaleric acid”, respectively. In some embodiments, any one or more of the foregoing reactant molecules of Formula (i) react with each other in the absence of a reactant molecule of Formula (ii) and in the presence of an amide synthetase enzyme to produce an amide-containing molecule of Formula (1). In other embodiments, any one or more of the foregoing reactant molecules of Formula (i) react with any one or more of reactant molecules within the scope of Formula (ii) in the presence of an amide synthetase enzyme to produce an amide-containing molecule of Formula (1).

In some embodiments, the reaction proceeds by reacting precisely or at least two, three, or four reactant molecules of Formula (i) that form an amide bond with each other in the presence of the amide synthetase enzyme. The end result is, respectively, a dimeric, trimeric, or tetrameric amide-containing molecule of Formula (1). In some embodiments, the precisely or at least two, three, or four reactant molecules of Formula (i) are selected from adipic acid (Ad), hexamethylenediamine (M), succinic acid(S), glutaric acid (G), pimelic acid (P), suberic acid (SU), azelaic acid (Z), sebacic acid (SE), muconic acid (MU), p-xylylenediamine (X), terephthalic acid (T), 1,4-cyclohexanedicarboxylic acid (CD), 2,5-furandicarboxylic acid (FD), cadaverine (C), 1,7-heptanediamine (HD), 1,8-octanediamine (O), 1,9-nonanediamine (ND), 1,10-decanediamine (DD), cis-1,4-cyclohexanediamine (N), 4-aminobutyrate (B), 5-aminovalerate (V), 6-aminohexanoic acid (H), 2-hydroxyglutaric acid (HG), α-ketoglutaric acid (K), 4-amino-2-hydroxybutanoic acid (E), and 4-amino-3-hydroxybutanoic acid (F).provided that the at least two, three, of four reactant molecules can form an amide bond with each other in the presence of the amide synthetase enzyme. For example, in some embodiments, one, two, or more reactant molecules selected from Ad, S, G, P, SU, Z, SE, MU, T, CD, FD, HG, and K is/are reacted with one, two, or more reactant molecules selected from M, X, C, HD, O, ND, DD, N, B, V, H, E, and F. Alternatively, in other embodiments, one, two, or more reactant molecules selected from M, X, C, HD, O, ND, DD, and N is/are reacted with one, two, or more reactant molecules selected from Ad, S, G, P, SU, Z, SE, MU, T, CD, FD, H, K, B, V, H, HG, E, and F.

In some embodiments, the reaction proceeds by reacting two reactant molecules of Formula (i) that form an amide bond with each other in the presence of the amide synthetase enzyme to result in a dimeric amide-containing molecule of the following formula:

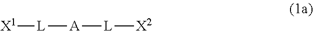

wherein X¹, X², L, and A are independently as defined earlier above. In some embodiments, the two reactant molecules of Formula (i) are selected from adipic acid (Ad), hexamethylenediamine (M), succinic acid (S), glutaric acid (G), pimelic acid (P), suberic acid (SU), azelaic acid (Z), sebacic acid (SE), muconic acid (MU), p-xylylenediamine (X), terephthalic acid (T), 1,4-cyclohexanedicarboxylic acid (CD), 2,5-furandicarboxylic acid (FD), cadaverine (C), 1,7-heptanediamine (HD), 1,8-octanediamine (O), 1,9-nonanediamine (ND), 1,10-decanediamine (DD), cis-1,4-cyclohexanediamine (N), 4-aminobutyrate (B), 5-aminovalerate (V), 6-aminohexanoic acid (H), 2-hydroxyglutaric acid (HG), α-ketoglutaric acid (K), 4-amino-2-hydroxybutanoic acid (E), and 4-amino-3-hydroxybutanoic acid (F), provided that the two reactant molecules can form an amide bond with each other in the presence of the amide synthetase enzyme. For example, in some embodiments, one reactant molecule selected from Ad, S, G, P, SU, Z, SE, MU, T, CD, FD, HG, and K is reacted with one reactant molecule selected from M, X, C, HD, O, ND, DD, N, B, V, H, E, and F. Alternatively, in other embodiments, one reactant molecule selected from M, X, C, HD, O, ND, DD, and N is reacted with one reactant molecule selected from Ad, S, G, P, SU, Z, SE, MU, T, CD, FD, H, K, B, V, H, HG, E, and F.

In particular embodiments, the two reactant molecules of Formula (i) have the following formulas:

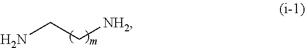

wherein m is an integer in a range of 1-11, and

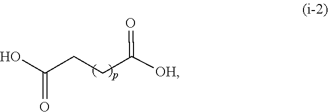

wherein p is an integer in a range of 1-11;

In different embodiments of the reaction between reactant molecules (i-1) and (i-2) above, m is an integer of 1, 2, 3, 4, 5, 6, 7, 8, 9, 10, or 11, or an integer within a range bounded by any two of the foregoing values, e.g., 1-10, 1-8, 1-6, 1-5, 1-4, 1-3, 2-11, 2-10, 2-8, 2-6, 2-5, 2-4, 2-3, 3-11, 3-10, 3-8, 3-6, 3-5, 4-11, 4-10, 4-8, 4-6, 4-5, 5-11, 5-10, 5-8, 5-6, 6-11, 6-10, or 6-8. In different embodiments, p is an integer of 1, 2, 3, 4, 5, 6, 7, 8, 9, 10, or 11, or an integer within a range bounded by any two of the foregoing values, e.g., 1-10, 1-8, 1-6, 1-5, 1-4, 1-3, 2-11, 2-10, 2-8, 2-6, 2-5, 2-4, 2-3, 3-11, 3-10, 3-8, 3-6, 3-5, 4-11, 4-10, 4-8, 4-6, 4-5, 5-11, 5-10, 5-8, 5-6, 6-11, 6-10, or 6-8. Any value (or range) provided above for m can be combined with any value (or range) provided above for p. For example m may be within a range of 4-11, 4-8, 4-6, or 4-5 and p may be within a range of 1-11, 1-10, 1-8, 1-6, 1-5, 1-4, or 1-3.

The reaction above between reactant molecules of Formulas (i-1) and (i-2) proceeds by reacting the two reactant molecules in the presence of an amide synthetase enzyme such as any of those described above, or more particularly, an SfaB or a homolog thereof, to result in a dimeric amide-containing molecule of the following sub-formula of Formula (1a):

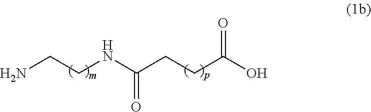

In Formula (1b) above, m and p can independently be any of the values or ranges thereof provided above, provided they are the same as the selections for m and p in Formulas (i-1) and (i-2), respectively.

In exemplary embodiments, the dimeric amide-containing molecule of the Formula (1b) is selected from the following:

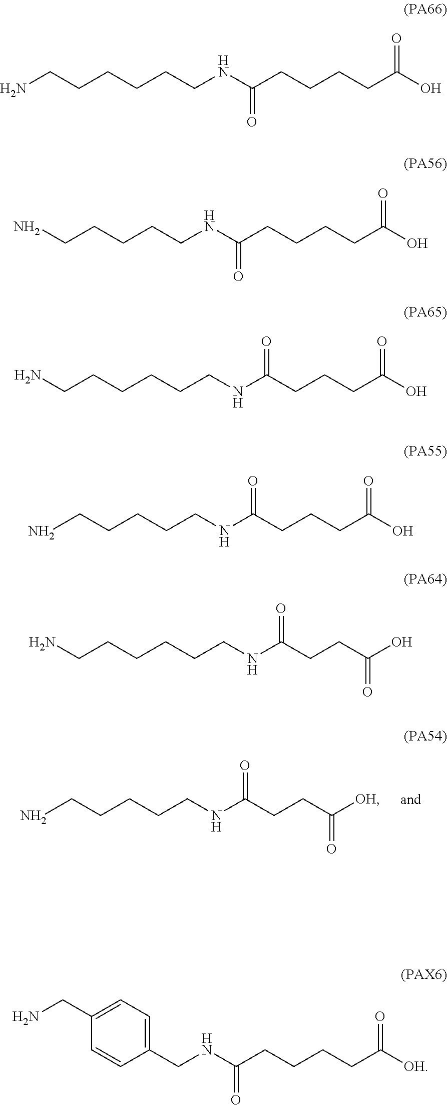

In some embodiments, the reaction above between reactant molecules of Formulas (i-1) and (i-2) to form the dimeric amide-containing molecule of the Formula (1b) proceeds by reacting the two reactant molecules in the presence an SfaB enzyme or a homolog thereof, such as AS9, AS17, AS41, AS3, or AS8, or a homolog of any of these homologs. The percent homology between SfaB and AS9 is 29.8%. The percent homology between SfaB and AS17 is 28.4%. The percent homology between SfaB and AS41 is 29.6%. The percent homology between SfaB and AS3 is 34.1%. The percent homology between SfaB and AS8 is 31.7%. Moreover, AS9, AS17, AS41, AS3, or AS8 are all approximately 30% identical to each other (e.g., AS9 is 29.8% identical to SfaB, AS8 is 31.7% identical to SfaB, and AS9 is 31.0% identical to AS8).

In other particular embodiments, the two reactant molecules of Formula (i) have the following formulas:

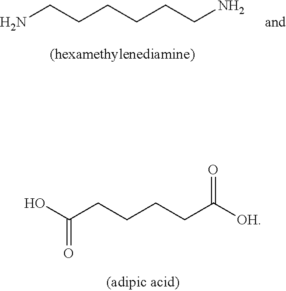 [SMILES: NCCCCCCN ; O=C(O)CCCCC(=O)O]

The reaction above between hexamethylenediamine and adipic acid reactant molecules proceeds by reacting the two reactant molecules in the presence of an amide synthetase enzyme such as any of those described above, or more particularly, an SfaB enzyme or a homolog thereof, such as any of those described above, to result in a PA66 dimeric amide-containing molecule of the following sub-formula of Formula (1a):

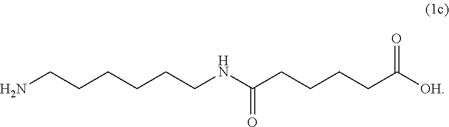 [SMILES: NCCCCCCNC(=O)CCCCC(=O)O]

In some embodiments, the reaction above between hexamethylenediamine and adipic acid reactant molecules to form the dimeric amide-containing molecule of the Formula (1c) proceeds by reacting the two reactant molecules in the presence an SfaB enzyme or a homolog thereof, such as AS9, AS17, AS41, AS3, or AS8, or a homolog of any of these homologs.

In other particular embodiments, the two reactant molecules of Formula (i) have the following formulas:

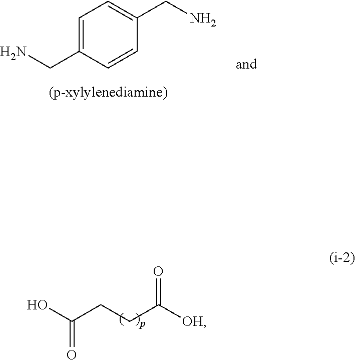

wherein p is an integer in a range of 1-11, such as any of the exemplary values or ranges provided above for p (e.g., 1-5).

The reaction above between p-xylylenediamine and reactant molecule of Formula (i-2), such as adipic acid (p=3), proceeds by reacting the two reactant molecules in the presence of an amide synthetase enzyme such as any of those described above, or more particularly, an SfaB enzyme or a homolog thereof, such as any of those described above, to result in a dimeric amide-containing molecule of the following sub-formula of Formula (1a):

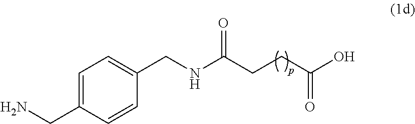

In some embodiments, the reaction above between p-xylylenediamine and reactant molecule of Formula (i-2) reactant molecules to form the dimeric amide-containing molecule of the Formula (1d) proceeds by reacting the two reactant molecules in the presence of an SfaB enzyme, such as SfaB, DesD, AcsA, or a homolog of any of those. In particular embodiments, the amide synthetase enzyme is AcsA.

In other embodiments, the reaction proceeds by reacting three or more molecules of Formula (i) that form an amide bond with each other in the presence of an amide synthetase enzyme to result in a trimeric or higher oligomeric amide-containing molecule of the Formula (1) wherein w is 1. The reaction may be conducted as a single-step reaction or a multi-step reaction. A single-step reaction refers to a reaction in which all reactant molecules necessary for producing the final amide molecule are reacted together in the same reaction vessel at one time in the presence of the amide synthetase enzyme. For a multi-step reaction, the same or a different enzyme may be employed for each step. As an example of a multi-step reaction, a dimeric amide molecule within Formula (1) may be formed by reacting two molecules of Formula (i) that form an amide bond with each other using a first amide synthetase enzyme selected from any of those disclosed herein, followed by reacting the dimeric amide molecule with a third molecule of Formula (i) using a second amide synthetase enzyme selected from any of those disclosed herein to form the trimeric amide molecule within Formula (1), wherein the first and second amide synthetase enzymes are the same or different. As another example, a dimeric amide molecule within Formula (1) may be formed by reacting two molecules of Formula (i) that form an amide bond with each other using a first amide synthetase enzyme selected from any of those disclosed herein, followed by reacting the dimeric amide molecule with a third molecule of Formula (i) using a second amide synthetase enzyme selected from any of those disclosed herein to form a trimeric amide molecule within Formula (1), followed by reacting the trimeric amide molecule with a fourth molecule of Formula (i) using a third amide synthetase enzyme selected from any of those disclosed herein to form a tetrameric amide molecule within Formula (1), wherein the first, second, and third amide synthetase enzymes are independently selected and may be the same or different. Alternatively, to form a tetrameric amide molecule within Formula (1), a dimeric amide molecule within Formula (1) may be formed by reacting two molecules of Formula (i) that form an amide bond with each other using a first amide synthetase enzyme selected from any of those disclosed herein, followed by reacting the dimeric amide molecule with two molecules of Formula (i) using a second amide synthetase enzyme, wherein the first and second amide synthetase enzymes are the same or different.

Some examples of possible combinations of three molecules of Formula (i) include: one reactant molecule selected from Ad, S, G, P, SU, Z, SE, MU, T, CD, FD, HG, and K and two reactant molecules selected from M, X, C, HD, O, ND, DD, N, B, V, H, E, and F; or two reactant molecules selected from Ad, S, G, P, SU, Z, SE, MU, T, CD, FD, HG, and K and one reactant molecule selected from M, X, C, HD, O, ND, DD, N, B, V, H, E, and F (or more particularly, M, X, C, HD, O, ND, DD, or N); or one reactant molecule selected from M, X, C, HD, O, ND, DD, and N and two reactant molecules selected from Ad, S, G, P, SU, Z, SE, MU, T, CD, FD, H, HG, K, B, V, H, E, and F; or two reactant molecules selected from M, X, C, HD, O, ND, DD, and N and one reactant molecule selected from Ad, S, G, P, SU, Z, SE, MU, T, CD, FD, H, K, B, V, H, E, and F (or more particularly, Ad, S, G, P, SU, Z, SE, MU, T, CD, or FD). Any of the above combinations or sub-combinations therein can function to produce a trimeric amide molecule of Formula (1), wherein the reaction is conducted as a single-step reaction or multi-step reaction, wherein a multi-step reaction employs the same or different enzyme for each step. Notably, in the case where two diamines are reacted with an amino-acid, a trimeric amide molecule cannot be generated due to a deficit in the number of carboxylic acid groups. However, if two diamines are reacted with an amino-acid in a single-step reaction, a mixture of dimeric amide molecules within the scope of Formula (1) can result.

Some examples of possible combinations of four molecules of Formula (i) include: one reactant molecule selected from Ad, S, G, P, SU, Z, SE, MU, T, CD, FD, HG, and K and three reactant molecules selected from M, X, C, HD, O, ND, DD, N, B, V, H, E, and F; or two reactant molecules selected from Ad, S, G, P, SU, Z, SE, MU, T, CD, FD, HG, and K and two reactant molecules selected from M, X, C, HD, O, ND, DD, N, B, V, H, E, and F; or three reactant molecules selected from Ad, S, G, P, SU, Z, SE, MU, T, CD, FD, HG, and K and one reactant molecule selected from M, X, C, HD, O, ND, DD, and N; or one reactant molecule selected from M, X, C, HD, O, ND, DD, and N and three reactant molecules selected from Ad, S, G, P, SU, Z, SE, MU, T, CD, FD, H, HG, and K; or two reactant molecules selected from M, X, C, HD, O, ND, DD, and N and two reactant molecules selected from Ad, S, G, P, SU, Z, SE, MU, T, CD, FD, H, HG, and K; or three reactant molecules selected from M, X, C, HD, O, ND, DD, and N and one reactant molecule selected from Ad, S, G, P, SU, Z, SE, MU, T, CD, FD, H, HG, and K. Any of the above combinations can function to produce a tetrameric amide molecule of Formula (1), wherein the reaction is conducted as a single-step reaction or multi-step reaction, wherein a multi-step reaction employs the same or different enzyme for each step.

In other embodiments, the reaction proceeds by reacting at least one molecule of Formula (i) with at least one molecule of Formula (ii) that form an amide bond with each other in the presence of an amide synthetase enzyme to result in a trimeric, tetrameric, or higher oligomeric amide-containing molecule of the Formula (1) wherein w is 1 (and x, y, and z may each be 0 or 1). In some embodiments, precisely one molecule of Formula (i) is reacted with precisely one molecule of Formula (ii). In that case, when v is 0 in Formula (ii), a trimeric amide-containing molecule of Formula (1) results; when v is 1 in Formula (ii), a tetrameric amide-containing molecule of Formula (1) results; and when v is 2 in Formula (ii), a pentameric amide-containing molecule of Formula (1) results. In other embodiments, two molecules of Formula (i) are reacted with one molecule of Formula (ii), in which case a tetrameric, pentameric, or hexameric amide-containing molecule of Formula (1) results when v is 0, 1, or 2, respectively. In other embodiments, one molecule of Formula (i) is reacted with two molecules of Formula (ii), in which case a pentameric amide-containing molecule of Formula (1) results when v is 0. In other embodiments, two molecules of Formula (i) are reacted with two molecules of Formula (ii), in which case a hexameric amide-containing molecule of Formula (1) results when v is 0. Any of the above reactions may be conducted as a single-step reaction or multi-step reaction, wherein a multi-step reaction employs the same or different enzyme for each step.

In other embodiments, the reaction proceeds by reacting two reactant molecules of Formula (ii) that form an amide bond with each other in the presence of the amide synthetase enzyme to result in a tetrameric or higher oligomeric amide-containing molecule of the Formula (1). The tetrameric or higher oligomeric amide-containing molecule of the Formula (1) can have the following formula:

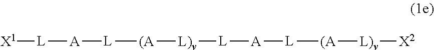

In the above Formula (1e), X¹, X², L, and A are independently as defined earlier above, and each v is independently 0 or 1. In some embodiments, both v are 0; in other embodiments, both v are 1; and in other embodiments, one v is 0 and one v is 1. In some embodiments, each reactant molecule of Formula (ii) is composed of a dimer independently formed from any two of: adipic acid (Ad), hexamethylenediamine (M), succinic acid (S), glutaric acid (G), pimelic acid (P), suberic acid (SU), azelaic acid (Z), sebacic acid (SE), p-xylylenediamine (X), terephthalic acid (T), 1,4-cyclohexanedicarboxylic acid (CD), 2,5-furandicarboxylic acid (FD), cadaverine (C), 1,7-heptanediamine (HD), 1,8-octanediamine (O), 1,9-nonanediamine (ND), 1,10-decanediamine (DD), cis-1,4-cyclohexanediamine (N), 4-aminobutyrate (B), 5-aminovalerate (V), 6-aminohexanoic acid (H), 2-hydroxyglutaric acid (HG), α-ketoglutaric acid (K), 4-amino-2-hydroxybutanoic acid (E), and 4-amino-3-hydroxybutanoic acid (F), provided that the two reactant molecules can form an amide bond with each other in the presence of the amide synthetase enzyme. The reaction can be conducted as a single-step reaction or multi-step reaction, wherein a multi-step reaction employs the same or different enzyme for each step. In particular embodiments, the amide synthetase enzyme used for reacting two molecules of Formula (ii) is a DesD homolog, such as any of those described above.

The present disclosure is also directed to the polymerized product of any one or more of the amide-containing molecules of Formula (1). When an amide-containing molecule of Formula (1) is used to produce a polymer, the amide-containing molecule can be considered a “building block” or a “unit” of the resulting polymer. In some embodiments, after producing one or more amide-containing molecules of Formula (1), the one or more amide-containing molecules of Formula (1) are polymerized by means well known in the art to form a polyamide polymer. The polyamide polymer can be generically represented by the following formula: -[NH-L-A-L-(A-L)_{w}-(A-L)ₓ-(A-L)_{y}-(A-L)_{z}-C(O)]ₙ- or -[(O)C-L-A-L-(A-L)_{w}-(A-L)ₓ-(A-L)_{y}-(A-L)_{z}-NH]ₙ-, wherein the variable n is typically at least 10, 20, 30, 40, 50, or 100.

As well known, amine and carboxylic acid groups (either on a single molecule or on different molecules) react with each other to form amide bonds by a condensation reaction. Methods for polymerizing amine-containing and carboxy-containing molecules to form polyamides are well known in the art. In some embodiments, a molecule of Formula (1) containing X¹ as NH₂ and X² as COOH (or X¹ as COOH and X² as NH₂) can be polymerized with itself to form a polymer containing repeating units of itself. As an example, a molecule of Formula (1) may have the following generic formula: H₂N-L¹-A-L₂-COOH, wherein L¹ and L² are different linkers L selected from any of the linkers described in this disclosure. Polymerization of the foregoing generic building block would result in a polyamide of the formula -(HN-L¹-A-L²-CO) n-, wherein the variable n is typically at least 10, 20, 30, 40, 50, or 100. As another example, a molecule of Formula (1) may have the following generic formula: H₂N-L¹-A-L²-A-L³-COOH, wherein L¹, L², and L³ are different linkers L selected from any of the linkers described in this disclosure. Polymerization of the foregoing generic building block would result in a polyamide of the formula -(HN-L¹-A-L²-A-L³-CO)ₙ-, wherein the variable n is typically at least 10, 20, 30, 40, 50, or 100.

In a case where the molecule of Formula (1) selects X¹ and X² as both NH₂ groups (i.e., is a diamine molecule), the diamine molecule is not polymerizable with itself to form a polyamide. For the diamine molecule to form a polyamide, the diamine molecule would need to be reacted with a dicarboxylic acid molecule (i.e., wherein X¹ and X² are both COOH), wherein the dicarboxylic acid molecule may be within or outside the scope of Formula (1) and may or may not be within the scope of Formula (i).

In a case where the molecule of Formula (1) selects X¹ and X² as both COOH groups (i.e., is a diacid molecule), the diacid molecule is not polymerizable with itself to form a polyamide. For the diacid molecule to form a polyamide, the diacid molecule would need to be reacted with a diamine molecule (i.e., wherein X¹ and X² are both NH₂), wherein the diamine molecule may be within or outside the scope of Formula (1) and may or may not be within the scope of Formula (i).

Notably, although the present disclosure primarily considers where molecules of Formula (1) are used as building blocks for forming polyamides, the molecules of Formula (1) can be used as building blocks to form other types of polymers. For example, a diamine of Formula (1) (i.e., wherein X¹ and X² are both NH₂) can be reacted with a diisocyanate molecule to form a polyurea. The diamine may alternatively be reacted with a dianhydride molecule to form a polyimide.

In some embodiments, the polymerized product is derived from an amide molecule of Formula (1d) and has the following formula:

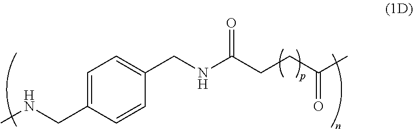

wherein p and n are independently as provided above.

Examples have been set forth below for the purpose of illustration and to describe certain specific embodiments of the invention. However, the scope of this invention is not to be in any way limited by the examples set forth herein.

### EXAMPLES

### Methods

In vitro Enzyme Activity Assays: A typical enzymatic reaction was carried in triplicate at 50 μL containing 100 mM HEPES (pH 8.0), 10 mM ATP, 10 mM MgCl_{2, 10} μM enzyme or 25 μL lysate, 5 mM carboxyl group-containing compounds and 5 mM amine group-containing compounds, and was incubated at 30° C. for 16 h with shaking at 200 rpm. The reaction was quenched by adding 50 μL methanol. The samples were centrifuged at 4,000 rpm for 10 min, and the supernatant was subjected to MS analysis to determine the formation of corresponding products. For the 50 ml reaction, 100 mM HEPES (pH 8.0), 50 mM ATP, 50 mM MgCl₂, 10 μM enzyme, 50 mM disodium succinic acid and 50 mM hexamethylenediamine were incubated at 30° C. for 16 h with shaking at 200 rpm. The reaction was quenched by adding 50 mL methanol. The samples were centrifuged at 4,000 rpm for 20 min, and the supernatant was subjected for product isolation.

Enzyme cascade Assays: For one-pot synthesis, the reaction was carried at 100 μL containing 100 mM HEPES (pH 8.0), 40 mM ATP, 40 mM MgCl₂, 10 μM DdaG, 20 μM DesD, 5 mM succinic acid, 5 mM hexamethylenediamine, and 5 mM 1,8-octanoic amino acid and was incubated at 30° C. for 16 h with shaking at 200 rpm. The reaction was quenched by adding 100 μL methanol. The samples were centrifuged at 4,000 rpm for 10 min, and the supernatant was subjected to MS analysis to determine the formation of corresponding products. For sequential synthesis, on day 1, the reaction was carried at 100 μL containing 100 mM HEPES (pH 8.0), 10 mM ATP, 10 mM MgCl₂, 10 μM DdaG, 5 mM succinic acid and 5 mM hexamethylenediamine and was incubated at 30° C. for 24 h with shaking at 200 rpm. On day 2, the reaction was stopped by boiling at 95° C. for 5 minutes and centrifuged at 4,000 rpm for 10 minutes. 25 μL of the supernatant was taken out for MS analysis. 100 mM HEPES (pH 8.0), 20 mM ATP, 10 mM MgCl₂, 20 μM DesD, 5 mM 1,8-diaminooctane was added to the remaining 75 μL supernatant and was incubated at 30° C. for 24 h with shaking at 200 rpm. On day 3, the reaction was stopped by boiling at 95° C. for 5 minutes and centrifuged at 4,000 rpm for 10 minutes. 25 μL of the supernatant was taken out for MS analysis. 100 mM HEPES (pH 8.0), 20 mM ATP, 10 mM MgCl₂, 10 μM SfaB, 5 mM glutaric acid was added to the remaining 75 μL supernatant and was incubated at 30° C. for 24 h with shaking at 200 rpm. On day 4, the reaction was stopped by boiling at 95° C. for 5 minutes and centrifuged at 4,000rpm for 10 minutes. 25 μL of the supernatant was taken out for MS analysis.

### Results and Discussion

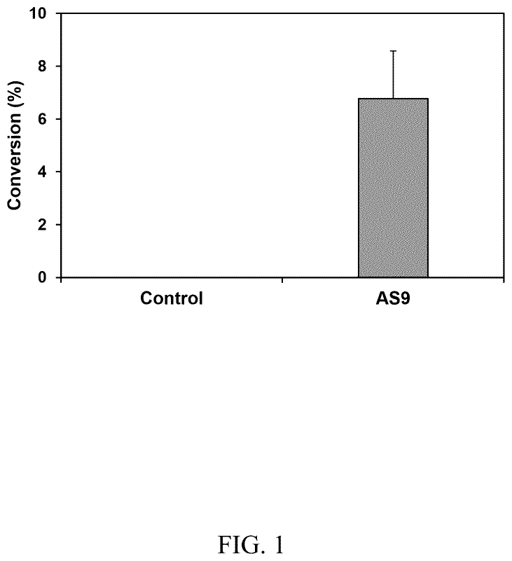

To explore additional enzymes for nylon diad synthesis, the amino acid sequence of SfaB (UniProt ID A0A2H4T912) was used as a query for three independent sequence searches using commercial software to find homologous sequences in nr70_10_Aug and alphafold_uniprot50 databases. A total of 20,531 unique sequences were acquired, of which 271 sequences appeared in all three searches. To investigate how changes in amino acid composition affect substrate binding, 51 potential binding-site residues in the SfaB AlphaFold structure were identified based on structural visualization and machine learning prediction by a graph neural network. 53 of the 271 sequences shared the same amino acid type as SfaB at more than 70% (36 sites) of the predicted binding sties in a sequence alignment. HHfilter software was then applied to the alignment using a minimum of 50% coverage with SfaB and a maximum pairwise sequence identity ranging from 30% to 95% and selected 12 maximally diverse homologs. The same approach was used for the alignment of the remaining 218 sequences which had higher amino acid variation at the predicted binding sites, and another 36 homologs were selected. Three of the 48 selected homologs were from BSL2 or BSL3 organisms and were replaced with three homologs from the 20,531-sequence pool with identity to SfaB lower than 30%. The pairwise sequence identities between the final 48 sequences are between 25% and 50%. Of the 48 selected SfaB homologs, purified AS9 showed the highest activity ligating adipic acid (A) and hexamethylenediamine (M) to PA66 monomer (MA) (FIG. 1).

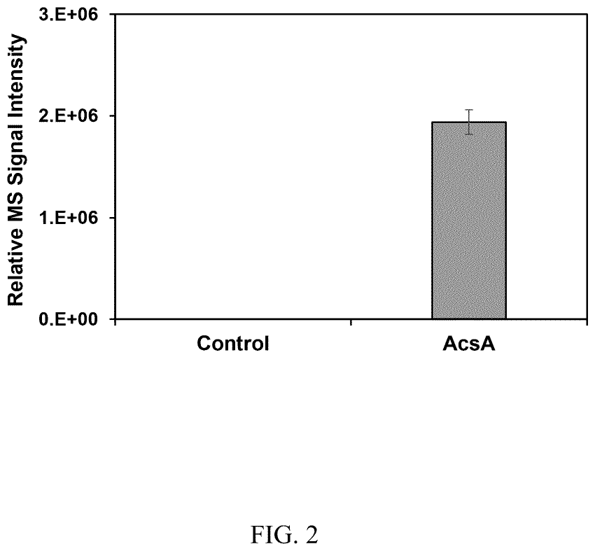

To identify more amide synthetases capable of coupling nylon relevant substrates, four NRPS-independent siderophore (NIS) synthetases, namely AsbA, AsbB, AcsA and DesD, were selected and purified as hexahistidine-tagged proteins to test their activities towards polymer relevant substrates in vitro. Results from IDOT/OPSI-MS analysis indicate that these NIS synthetases were active with non-native substrates and exhibited a broadened substrate scope compared to previously reported enzymes. Notably, AcsA demonstrated approximately 7-fold higher activity than SfaB when ligating adipic acid (A) and p-xylylenediamine (X) to form the XA diad (FIG. 2).

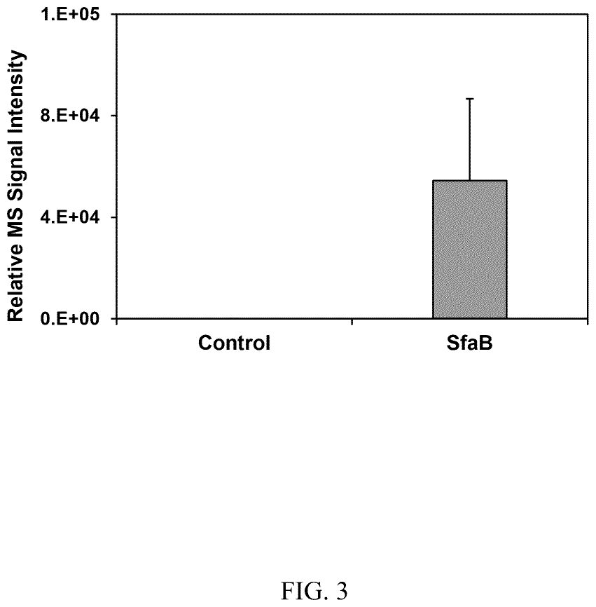

Although it was shown above that amide synthetases can form nylon-relevant diads, the synthesis of novel multi-component copolyamides will require generating longer oligoamides. Therefore, next experiments tested whether amide diads can serve as substrates for triad formation. The diad MA (i.e., PA66 monomer) was chemically synthesized and testing was done to determine whether the selected enzymes could ligate these diads with other polymer-relevant substrates. It was herein found that SfaB can ligate the synthesized ω-amino acid diad MA with diacid substrates (i.e., adipic acid (A)) to make diacid triads (FIG. 3).

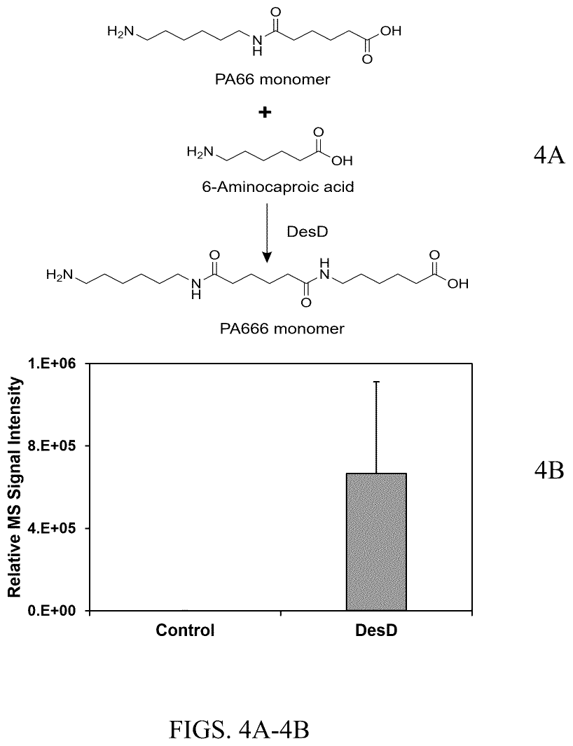

In addition, DesD exhibited distinctive activities in ligating ω-amino acid diads with ω-amino acid monads to form diamine or ω-amino acid triads. Specifically, using I.DOT/OPSI-MS, it was herein demonstrated that DesD can ligate an ω-amino acid diad (i.e. the PA66 monomer, MA) with an ω-amino acid (e.g. 6-aminohexanoic acid, H) to form an ω-amino acid triad (FIGS. 4A-4B). When ligating an ω-amino acid diad with an ω-amino acid monad, both substrates are bifunctional and can serve as either a carboxylic donor or an amine acceptor, thus leading to two possible regioisomers. For example, the reaction of MA and H could yield MAH or HMA. To determine the regioselectivity of DesD in ω-amino acid triad synthesis, both possible triad standards, MAH and HMA, were chemically synthesized and MS² was used to identify their unique fragment ions. The MS² spectrum of the enzymatic product matched only the MAH standard, indicating that DesD selectively forms a single ω-amino acid triad product.

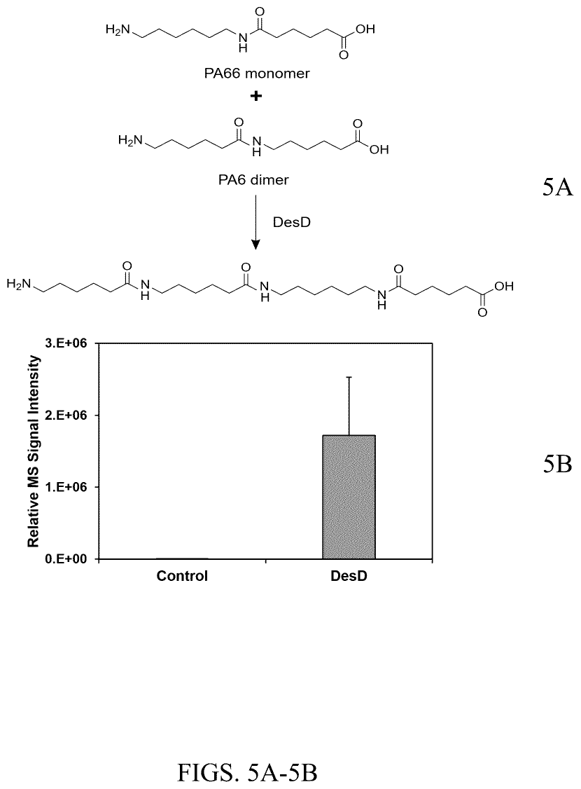

Motivated by the success of forming the amide triad, the use of these enzymes to form amide tetrads was assessed. The results show that DesD can regioselectively dimerize synthesized and commercial diads (i.e., PA6 dimer HH and the PA66 monomer MA) to form ω-amino acid tetrads HHMA (FIGS. 5A-5B), thus highlighting the versatile potential of DesD in synthesizing complex, sequence-controlled oligoamides.

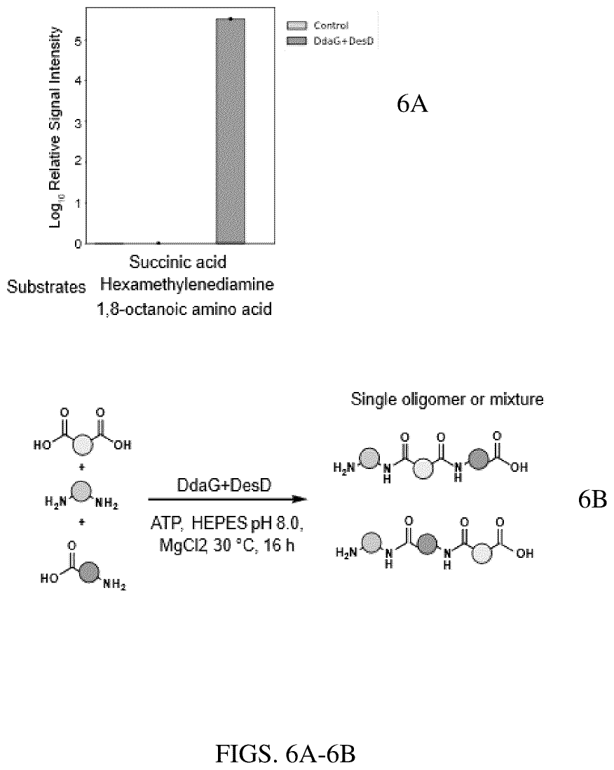

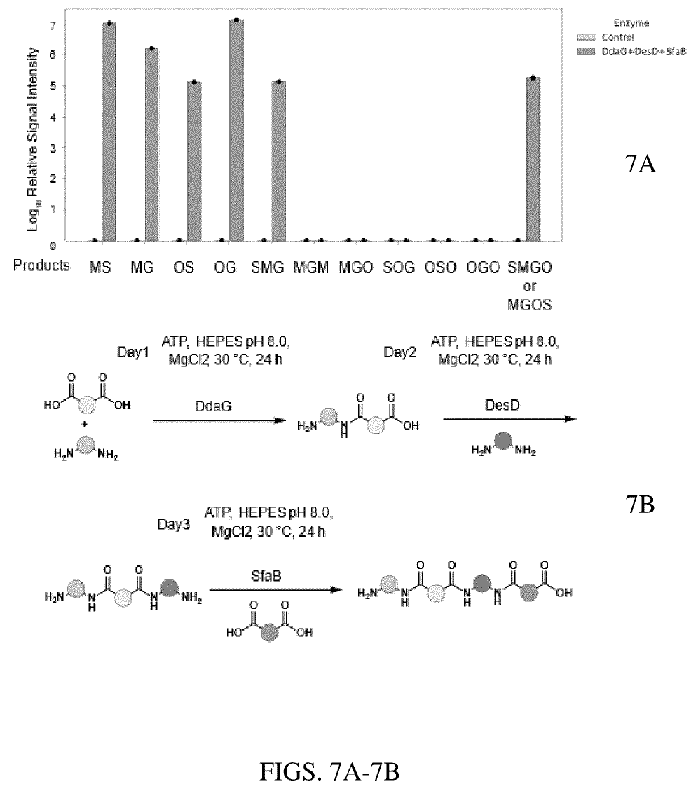

The high catalytic activity of DdaG and SfaB with S and G across multiple diamines, combined with DesD's unique ability to form diverse amide oligomers, led to further experiments aimed at exploring an enzymatic cascade for sequenced oligomer synthesis directly from unprotected substrates. To test the feasibility of this approach, a two-enzyme model cascade was first constructed. Since DdaG can actively couple S and M, and DesD can ligate ω-amino acid diads with ω-amino acid monads, a simultaneous one-pot reaction was first conducted to assess whether DdaG and DesD could cooperatively form an ω-amino acid triad from S, M, and 8-aminooctanoic acid (R). I.DOT/OPSI-MS analysis confirmed the formation of the target ω-amino acid amide triad (SMR), thus demonstrating the feasibility of using the two-enzyme cascade (FIGS. 6A-6B). Encouraged by the ω-amino acid triad formation, further efforts studied a one-pot reaction with DdaG, SfaB and DesD to form an ω-amino acid amide tetrad using S, G, M, and 1,8-diaminooctane (O). However, no tetrads were detected. Subsequently, a one-pot sequential reaction strategy was developed. First, DdaG was used to ligate S and M to form the MS diad. After 24 h, DesD was added to ligate the newly-synthesized MS diad with O to form a diamine triad, MSO. After a further 24 h, SfaB was added to couple the diamine triad MSO with G. After a total reaction time of 72 h, the final tetrad containing succinate, glutarate, hexamethylenediamine and 1,8-diaminooctane was detected by I.DOT/OPSI-MS (FIGS. 7A-7B). In addition to the desired product, the formation of a range of side products, including MS, MG, OS, OG, and SMG, was observed, likely due to excess substrate and limited substrate specificity. This observation highlights the need for future enzyme engineering to enhance enzyme efficiency and substrate specificity towards sequence-controlled oligoamide synthesis. Nevertheless, this sequential cascade provides an enzymatic pathway for a protecting-group-free biocatalytic route to diverse oligoamides from polymer-relevant substrates.

While there have been shown and described what are at present considered the preferred embodiments of the invention, those skilled in the art may make various changes and modifications which remain within the scope of the invention defined by the appended claims.

What is claimed is:

1. A method for producing amide-containing molecules of the formula: 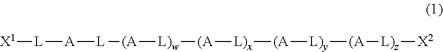
wherein:
X¹ and X² are independently selected from NH₂ and COOH;
L is independently, in each instance, selected from hydrocarbon linkers containing 1-12 carbon atoms optionally containing one or more heteroatoms selected from O, N, S, and halogen atoms, and are the same or different;
A independently represents in each instance an —NHC(O)— or —C(O)NH— linkage;
W, x, y, and z are independently 0 or 1;
the method comprising reacting at least two reactant molecules that form an amide bond with each other via X¹ and X² groups, in the presence of an amide synthetase enzyme, wherein the at least two reactant molecules are independently selected from the following formulas: 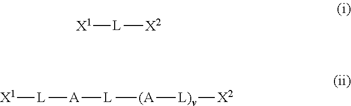
wherein v is 0, 1, or 2 and the at least two reactant molecules undergoing the reaction are selected from Formula (i), or from Formula (ii), or from at least one molecule of Formula (i) and at least one molecule of Formula (ii);
wherein the reaction of the at least two reactant molecules above results in an amide-containing molecule of Formula (1).

2. The method of claim 1, wherein the amide synthetase enzyme is selected from the group consisting of DdaG, SfaB, DesD, AsbA, AsbB, AcsA, and homologs thereof.

3. The method of claim 1, wherein at least one L in Formula (1) is selected from linear or branched alkylene linkers containing 1-12 carbon atoms and optionally one or more heteroatoms selected from O, N, S, and halogen atoms.

4. The method of claim 1, wherein at least one L in Formula (1) includes a ring and optionally contains one or more heteroatoms selected from O, N, S, and halogen atoms.

5. The method of claim 1, wherein the at least one reactant molecule of Formula (i) is selected from the group consisting of adipic acid (Ad), hexamethylenediamine (M), succinic acid (S), glutaric acid (G), pimelic acid (P), suberic acid (SU), azelaic acid (Z), sebacic acid (SE), muconic acid (MU), p-xylylenediamine (X), terephthalic acid (T), 1,4-cyclohexanedicarboxylic acid (CD), 2,5-furandicarboxylic acid (FD), cadaverine (C), 1,7-heptanediamine (HD), 1,8-octanediamine (O), 1,9-nonanediamine (ND), 1,10-decanediamine (DD), cis-1,4-cyclohexanediamine (N), 4-aminobutyrate (B), 5-aminovalerate (V), 6-aminohexanoic acid (H), 2-hydroxyglutaric acid (HG), α-ketoglutaric acid (K), 4-amino-2-hydroxybutanoic acid (E), and 4-amino-3-hydroxybutanoic acid (F).

6. The method of claim 1, wherein the reaction proceeds by reacting at least two reactant molecules of Formula (i) that form an amide bond with each other in the presence of the amide synthetase enzyme.

7. The method of claim 6, wherein the at least two reactant molecules of Formula (i) are selected from the group consisting of adipic acid (Ad), hexamethylenediamine (M), succinic acid (S), glutaric acid (G), pimelic acid (P), suberic acid (SU), azelaic acid (Z), sebacic acid (SE), muconic acid (MU), p-xylylenediamine (X), terephthalic acid (T), 1,4-cyclohexanedicarboxylic acid (CD), 2,5-furandicarboxylic acid (FD), cadaverine (C), 1,7-heptanediamine (HD), 1,8-octanediamine (O), 1,9-nonanediamine (ND), 1,10-decanediamine (DD), cis-1,4-cyclohexanediamine (N), 4-aminobutyrate (B), 5-aminovalerate (V), 6-aminohexanoic acid (H), 2-hydroxyglutaric acid (HG), α-ketoglutaric acid (K), 4-amino-2-hydroxybutanoic acid (E), and 4-amino-3-hydroxybutanoic acid (F).

8. The method of claim 1, wherein the reaction proceeds by reacting two reactant molecules of Formula (i) that form an amide bond with each other in the presence of the amide synthetase enzyme to result in a dimeric amide-containing molecule of the following formula: 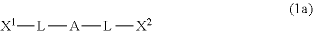
wherein X¹, X², L, and A are as defined in claim 1.

9. The method of claim 8, wherein the two reactant molecules of Formula (i) are selected from the group consisting of adipic acid (Ad), hexamethylenediamine (M), succinic acid (S), glutaric acid (G), pimelic acid (P), suberic acid (SU), azelaic acid (Z), sebacic acid (SE), muconic acid (MU), p-xylylenediamine (X), terephthalic acid (T), 1,4-cyclohexanedicarboxylic acid (CD), 2,5-furandicarboxylic acid (FD), cadaverine (C), 1,7-heptanediamine (HD), 1,8-octanediamine (O), 1,9-nonanediamine (ND), 1,10-decanediamine (DD), cis-1,4-cyclohexanediamine (N), 4-aminobutyrate (B), 5-aminovalerate (V), 6-aminohexanoic acid (H), 2-hydroxyglutaric acid (HG), α-ketoglutaric acid (K), 4-amino-2-hydroxybutanoic acid (E), and 4-amino-3-hydroxybutanoic acid (F).

10. The method of claim 8, wherein the two reactant molecules of Formula (i) have the following formulas: 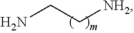
wherein m is an integer in a range of 1-11, and 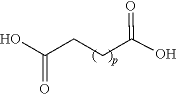
wherein p is an integer in a range of 1-11;
wherein the reaction proceeds by reacting the above two reactant molecules of Formula (i) in the presence of an amide synthetase enzyme to result in a dimeric amide-containing molecule of the following sub-formula of Formula (1a): 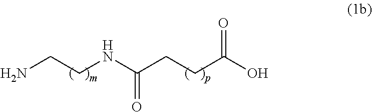
wherein the amide synthetase enzyme is SfaB or a homolog thereof.

11. The method of claim 10, wherein m is an integer in a range of 4-8 and p is an integer in a range of 1-6.

12. The method of claim 10, wherein the dimeric amide-containing molecule of the Formula (1b) is selected from the following:

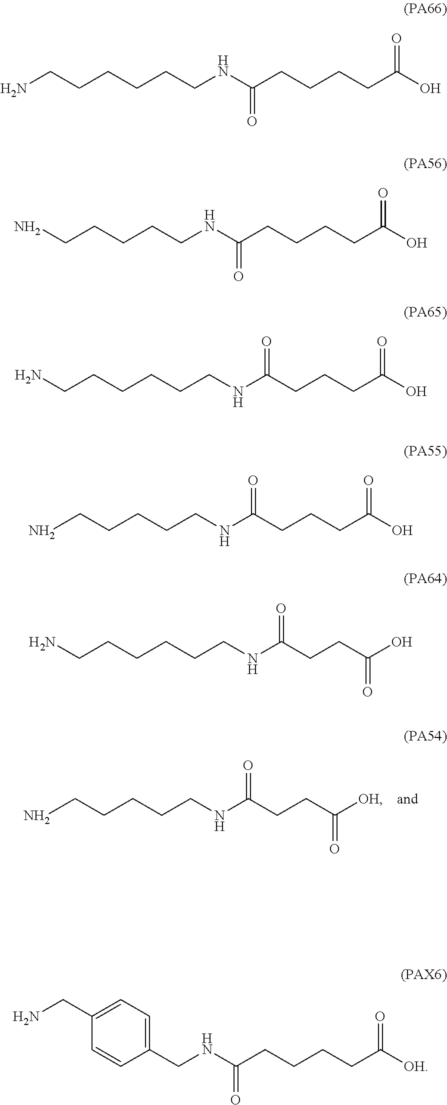

13. The method of claim 10, wherein the homolog of SfaB is selected from AS9 or homolog thereof, AS17 or homolog thereof, AS41 or homolog thereof, AS3 or homolog thereof, or AS8 or homolog thereof.

14. The method of claim 8, wherein the two reactant molecules of Formula (i) have the following formulas: 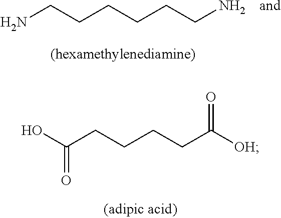 [SMILES: NCCCCCCN ; "and" ; O=C(O)CCCCC(=O)O]
wherein the reaction proceeds by reacting the above two reactant molecules of Formula (i) in the presence of an amide synthetase enzyme to result in a PA66 dimeric amide-containing molecule of the following sub-formula of Formula (1a): 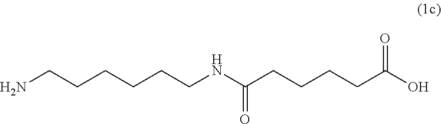 [SMILES: NCCCCCCNC(=O)CCCCC(=O)O]
wherein the amide synthetase enzyme is a homolog of SfaB.

15. The method of claim 14, wherein the homolog of SfaB is selected from AS9 or homolog thereof, AS17 or homolog thereof, AS41 or homolog thereof, AS3 or homolog thereof, or AS8 or homolog thereof.

16. The method of claim 8, wherein the two reactant molecules of Formula (i) have the following formulas: 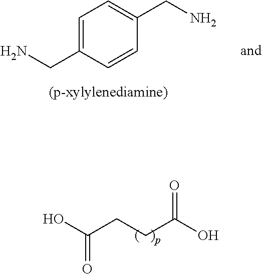
wherein p is an integer in a range of 1-11;
wherein the reaction proceeds by reacting the above two reactant molecules of Formula (i) in the presence of an amide synthetase enzyme to result in a dimeric amide-containing molecule of the following Formula (1b): 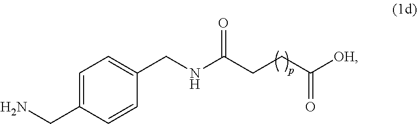
wherein the amide synthetase enzyme is selected from any one of SfaB or homolog thereof, DesD or homolog thereof, AcsA or homolog thereof.

17. The method of claim 16, wherein the amide synthetase enzyme is AcsA or homolog thereof.

18. The method of claim 1, wherein the reaction proceeds by reacting three or more molecules of Formula (i) that form an amide bond with each other in the presence of an amide synthetase enzyme to result in a trimeric or higher oligomeric amide-containing molecule of the Formula (1) wherein w is 1, and wherein the reaction is conducted as a single-step reaction or multi-step reaction, wherein a multi-step reaction employs the same or different catalyst for each step.

19. The method of claim 1, wherein the reaction proceeds by reacting at least one molecule of Formula (i) with at least one molecule of Formula (ii), with v=0, 1, or 2, that form an amide bond with each other in the presence of an amide synthetase enzyme to result in a trimeric or higher oligomeric amide-containing molecule of the Formula (1) wherein w is 1, and wherein the reaction is conducted as a single-step reaction or multi-step reaction, wherein a multi-step reaction employs the same or different catalyst for each step.

20. The method of claim 1, wherein the reaction proceeds by reacting at least one molecule of Formula (i) with at least one molecule of Formula (ii) that form an amide bond with each other in the presence of an amide synthetase enzyme to result in a tetrameric or higher oligomeric amide-containing molecule of the Formula (1) wherein w and x are each 1, and wherein the reaction is conducted as a single-step reaction or multi-step reaction, wherein a multi-step reaction employs the same or different catalyst for each step.

21. The method of claim 1, wherein the reaction proceeds by reacting two or more molecules of Formula (ii) that form an amide bond with each other in the presence of an amide synthetase enzyme to result in a tetrameric or higher oligomeric amide-containing molecule of the Formula (1) wherein w and x are each 1, and wherein the reaction is conducted as a single-step reaction or multi-step reaction, wherein a multi-step reaction employs the same or different catalyst for each step.

22. The method of claim 21, wherein the amide synthetase enzyme is a DesD homolog.

23. The method of claim 1, further comprising a subsequent step of polymerizing the amide-containing molecule of Formula (1) to form a polyamide polymer.

24. An amide-containing compound having the following formula: 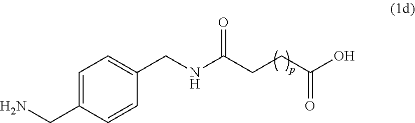
wherein p is an integer in a range of 1-11.

25. The amide-containing compound of claim 24, wherein p is an integer in a range of 1-5.

26. A polyamide polymer having the following formula: 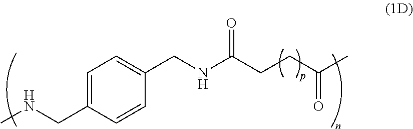
wherein p is an integer in a range of 1-11 and n is an integer of at least 10.
